(19) United States
(12) Patent Application Publication
(10) Pub. No.: US 2001/0026937 A1
(43) Pub. Date: Oct. 4, 2001

(54) Monocyte-derived dendritic cell subsets

(76) Inventors: Juha Punnonen, Palo Alto, CA (US); Chia-Chun J. Chang, Los Gatos, CA (US)

Correspondence Address: LAW OFFICES OF JONATHAN ALAN QUINE, P O BOX 458, ALAMEDA, CA, 94501

(21) Appl. No.: 09/760,388

(22) Filed: Jan. 10, 2001

Related U.S. Application Data
(60) Provisional application No. 60/175,552, filed on Jan. 11, 2000.
(60) Provisional application No. 60/181,957, filed on Feb. 10, 2000.

(51) Int. Cl. A61K 48/00; A01N 63/00; C12N 5/00; C12N 5/02

(52) U.S. Cl. 435/366; 435/325; 435/373; 424/93.21

(57) ABSTRACT

A novel subset of monocyte-derived dendritic cells are provided. Methods for producing these monocyte-derived dendritic cells and compositions comprising the dendritic cells of the invention are also provided. Methods for inducing an immune response to an antigen of interest using the dendritic cells of the invention are provided. Also provided are methods for therapeutically or prophylactically treating a disease in a subject suffering from the disease using the dendritic cells.

### CROSS-REFERENCES TO RELATED APPLICATIONS

This application claims priority to and benefit of U.S. Provisional Patent Application Ser. Nos. 60/175,552, filed on Jan. 11, 2000, and No. 60/181,957, filed on Feb. 10, 2000, the disclosures of each of which is incorporated herein in their entirety for all purposes.

### STATEMENT AS TO RIGHTS TO INVENTIONS MADE UNDER FEDERALLY SPONSORED RESEARCH AND DEVELOPMENT

This work was supported in part by a grant from the Defense Advanced Research Projects Agency (DARPA) (Grant No. N65236-98-1-5401). The Government may have certain rights in this invention.

### COPYRIGHT NOTIFICATION

Pursuant to 37 C.F.R. 1.71(e), Applicants note that a portion of this disclosure contains material which is subject to copyright protection. The copyright owner has no objection to the facsimile reproduction by anyone of the patent document or patent disclosure, as it appears in the Patent and Trademark Office patent file or records, but otherwise reserves all copyright rights whatsoever.

### FIELD OF THE INVENTION

The invention relates to the field of immunology. More particularly, the invention relates to the generation of a novel subtype of dendritic cells and to their use as antigen presenting cells.

### BACKGROUND OF THE INVENTION

Dendritic cells (DC) are the most potent antigen-presenting cells (APC) known to date, and their interaction with T cells is a key event in the early stages of a primary immune response. DC express high levels of Major Histocompatibility (MHC) molecules and costimulatory molecules, such as CD40, CD80, and CD86. DC also produce high levels of T cell cytokines, including the interleukins IL-6, IL-8, IL-10, and IL-12 (Cella et al. (1997) Curr Opin Immunol 9:10; Banchereau and Steinman (1998) Nature 392:245). These properties, combined with the efficient capture of antigens (Ags) by immature DC, allow DC to efficiently present antigenic peptides and costimulate antigen-specific näive T cells (Cella et al. (1997) Curr Opin Immunol 9:10; Banchereau and Steinman (1998) Nature 392:245).

The interaction of T cells with APC plays an important role in promoting and directing T helper (Th) cell differentiation. For example, methods have been proposed for activating T cells in vitro by exposure to antigen presenting dendritic cells, see, e.g., WO 94/02156 “METHODS FOR USING DENDRITIC CELLS TO ACTIVATE T CELLS” by Engleman et al., published Feb. 3, 1994; WO 94/21287 “PEPTIDE COATED DENDRITIC CELLS AS IMMUNOGENS” by Berzofsky et al., published Sep. 29, 1994; and WO 95/43638 “METHODS FOR IN VIVO T CELL ACTIVATION BY ANTIGEN-PULSED DENDRITIC CELLS” by Engleman et al., published Dec. 21, 1995.

In addition, several molecules, including membrane-bound costimulatory molecules, cytokines, and the MHC-peptide complex, have been implicated in determining the phenotype of differentiated T cells. The duration and intensity of T cell receptor engagement are important in triggering T cell responses (Viola and Lanzavecchia (1996) Science 273:104; Carballido et al. (1997) Eur J Immunol 27:515), but the cytokine environment plays the most important role in determining the resulting cytokine production profile and effector function of the differentiated T helper cells (O'Garra (1998) Immunity 8:275; Coffman et al. (1999) Curr Top Microbiol Inmunol 238:1).

IL-12 directs T helper 1 (Th1) differentiation in both human and murine systems (Hsieh et al. (1993) Science 260:547; Manetti et al. (1993) J Exp Med 177:1199; Simpson et al. (1988) J Exp Med 177:1199), whereas IL-4 mediates Th2 cell differentiation (Swain et al. (1990) J Immunol 145:3796; Le Gros et al. (1990) J Exp Med 172:921; Shimoda et al (1996) Nature 380:630). Moreover, TGF-β favors differentiation of Th3 cells (Chen et al. (1994) Science 265:1237), and IL-10 has been shown to skew T cell responses toward T regulatory cells that produce high levels of IL-10 and inhibit antigen-specific T cell responses (Groux et al. (1997) Nature 389:737; Asseman et al. (1999) J Exp Med 190:995).

DC are known for their capacity to produce high levels of IL-12 upon activation (Macatonia et al. (1995) J Immunol 154:5071; Koch et al. (1996) J Exp Med 184:741), whereas IL-4 production is undetectable. Therefore, the mechanisms that regulate the initial steps in Th2 cell differentiation have remained controversial.

NK1.1⁺ T cells have been shown to produce high levels of IL-4 following activation, which was also essential for the induction of a Th2 response and IgE isotype switching in vivo (Yoshimoto et al. (1995) Science 270:1845). However, IL-12 induces interferon-γ (IFN-γ) production even by highly polarized Th2 cells (Mocci and Coffman (1995) J Immunol 154:3779), and T cell precursors have the capacity to develop into either Th1 or Th2 under the appropriate conditions (Kamogawa (1993) Cell 75:985; Sad and Mossman (1995) J Immunol 153:3514). Therefore, it appears that induction of Th2 responses involves a relative absence of IL-12 during antigen presentation, further indicating that the cytokine synthesis profile of the APC plays an important role in determining the phenotype of the Th cells.

Thus, while it is evident that DC play a role in determining the effector function and activation status of T cells, problems remain in their use as immunotherapeutic agents. For example, although a biased Th1 response may be desirable for certain applications, the ability to influence a T cell response toward a Th2 phenotype has not been possible using dendritic cells in vitro. In addition, DC have proven refractory to transfection with exogenous gene sequences limiting their utility in many applications. The present invention addresses these and other difficulties in generating and using DC.

### SUMMARY OF THE INVENTION

The present invention provides a novel subset of monocyte-derived dendritic cells, designated “mDC2.” These cells are morphologically indistinguishable from classical or conventional known dendritic cells, herein designated “mDC1,” but differ significantly in a number of important characteristics, including marker expression and cytokine production profiles. In contrast to mDC1, which stimulate Th1 differentiation of immature T helper cells, mDC2 enhance development of T cells along the Th0/Th2 pathway. In addition, mDC2 demonstrate an increased amenability to transfection by exogenous DNA molecules, improving their capacity to act as antigen presenting cells in a variety of experimental applications, methods for the therapeutic and prophylactic treatment of diseases or disorders, particularly to antigens associated with diseases or disorders, genetic (e.g., DNA) vaccine or protein vaccine applications, immunotherapies, and gene therapy.

In one aspect, the invention provides methods for the differentiation of mononuclear cells or monocytes, particularly monocytes derived from peripheral blood or bone marrow, into antigen presenting cells (APC) in interleukin-4 (IL-4), granulocyte macrophage colony stimulating factor (GM-CSF), and a culture medium supplemented with insulin, transferrin, and various lipids, including linoleic acid, oleic acid, and palmitic acid. In preferred embodiments, the APC are dendritic cells. The dendritic cells of the invention (mDC2) are distinguishable from conventional dendritic cells (mDC1), in that they do not express substantially the cell surface marker CD1a, and in that they exhibit an altered cytokine production profile relative to mDC1. The cytokine production profile of these CD1a⁻ DC of the invention (mDC2) is characterized by a lack of IL-12 production and production of a higher level of IL-10 than is observed with the conventional mDC1.

In one embodiment, the culture medium is Iscove's modified Dulbecco's medium (IMDM). In some such embodiments, the IMDM is further supplemented with insulin, human transferrin, linoleic acid, oleic acid, palmitic acid, bovine serum albumin, and 2-amino ethanol. The medium may also be supplemented with IL-4 and GM-CSF (granulocyte-macrophage colony stimulating factor). In a preferred embodiment, the culture medium is Yssel's medium as described in Yssel et al. (1984) J Immunol Methods 72(1):219. All such media may also be supplemented with fetal bovine serum, glutamine, penicillin, and streptomycin.

The monocytes provided in the methods of the invention are derived from a human or non-human animal by using various methods, e.g., by leukopharesis or bone marrow aspiration. In some embodiments, a source of monocytes is depleted of alternative cell types by negative depletion of T, B and NK (natural killer) cells from density gradient preparations of mononuclear cells. In one embodiment, mononuclear cells are derived from buffy coat preparations of peripheral blood. In a preferred embodiment, depletion of T, B, and NK cells is performed using immunomagnetic beads.

The invention further provides methods for the maturation of APC in a comprising culturing the APC in medium containing anti-CD40 monoclonal antibody (mAb) followed by culture in the presence of lipopolysaccharide (LPS) and IFN-γ.

In some embodiments, the mDC2 cells of the invention are transfected with exogenous DNA molecules which encode one or more antigens, thereby producing mDC2 cells which preferentially present one or more antigens of interest. Alternatively, at least one antigen may be externally loaded by supplying the mDC2 cell with a source of exogenous peptide. In preferred embodiments, the at least one antigen is derived from a tumor cell, a bacterially-infected cell, a virally-infected cell, a parasitically-infected cell, or a target cell of an autoimmune response.

In addition, the invention provides for methods for inducing an immune response in a subject, comprising administering an APC of the invention to a subject, including, e.g., a human or other animal subject. The APC may be a dendritic cell of the invention, such as an mDC2, that displays at least one antigen of interest on its surface. An amount of the dendritic cell displaying the at least one antigen sufficient to induce an immune response is administered to the subject. Another aspect of the invention provides methods for the activation of T cells in vivo, ex vivo, or in vitro using the APC of the invention. These activated T cells are optionally administered or transferred to a subject.

The invention also provides for cell cultures containing monocytes, dendritic cells, and/or partially differentiated cells committed to a monocyte-dendritic cell differentiation pathway. In a preferred embodiment, any or all of these cells are present in Yssel's medium supplemented with IL-4 and GM-CSF.

In another aspect, the invention provides for antigen presenting cells produced by the methods of the invention. In some embodiments, the APC is a dendritic cell. The dendritic cells of the invention are characterized by a lack of IL-12 production and/or a high level of IL-10 production. In some embodiments, such dendritic cells are mDC2, as described herein and in greater detail below.

Another aspect of the invention relates to the differentiation of T cells into the Th0/Th2 subtype induced by the APC of the invention. Induction of T cell differentiation is most significantly based on exposure to cytokines. Conventional dendritic cells induce Th1, whereas the mDC2 of the invention induce, promote, or favor Th0/Th2 differentiation.

Another embodiment of the invention relates to the induction of an immune response by administering or transferring mDC2 cells, which present or display at least one antigen of interest, into a subject. The at least one antigen, which is preferably derived from a protein differentially expressed on a tumor cell or an infected cell, is optionally loaded onto the surface or expressed on or at the surface of the APC.

In another aspect, the invention provides for compositions containing mDC2 which display or present at least one antigen of interest. Such compositions can be used for therapeutic and prophylactic treatment of a variety of diseases, such as for example, tumors, cancers, or infectious diseases or for prophylactic or therapeutic administrations, such as in vaccine or gene therapy applications.

In yet another aspect, the invention provides a method of inducing differentiation of T cells, the method comprising: co-culturing a population of T cells with population of CD1a⁻ antigen presenting cells (APC), thereby inducing or promoting differentiation of said T cells.

In another aspect, the invention provides a differentiated antigen presenting cell (APC), which differentiated APC does not express CD1a cell surface marker.

### BRIEF DESCRIPTION OF THE FIGURES

FIG. 1. A bar graph illustrating IL-12 production by DC generated under different culture conditions.

FIG. 2. A series of histograms illustrating the characterization of the cell surface phenotype of freshly isolated monocytes (A), DC differentiated in the presence of IL-4 and GM-CSF in Yssel's medium (B), or RPMI (C).

FIG. 3. A series of bar graphs depicting cytokine production profiles of mDC1 and mDC2.

FIG. 4. Flow cytometry scatter plots demonstrating maturation of mDC1 (A) and mDC2 (B) into CD83+.

FIG. 5. Line graphs depicting proliferative response in mixed lymphocyte reactions (MLR) induced by (A) immature and (B) mature mDC1 (filled squares) and mDC2 (open circles).

FIG. 6. A series of bar graphs illustrating T cell differentiation in the presence of mDC1 and mDC2: (A) IFN-γ production; (B) IL-5 (filled bars) and IL-13 (open bars) production; (C) ratio of IFN-γ/IL-5 production; and (D) ratio of IFN-γ/IL-13 production.

FIG. 7. Scatter plots illustrating transfection frequencies of mDC1 with (A) negative control (control vector with no promoter) and (B) naked DNA; and mDC2 with (C) negative control and (D) naked DNA.

### DETAILED DISCUSSION

Dendritic cells (DC) are highly effective antigen presenting cells that are capable of priming and stimulating T cell responses to a wide variety of antigens. As such, they play a critical role in the immune response against tumors as well as numerous bacterial and other pathogens. For a detailed discussion of dendritic cells as well as numerous other topics of interest in the context of the present invention, see, e.g., Paul (1998) Fundamental Immunology, 4^{th} edition, Lippincott-Raven, Philadelphia (hereinafter “Paul”).

The present invention provides for unique subtypes of monocyte-derived antigen presenting dendritic cells which are characterized by a distinct cell surface marker profile and cytokine production profile, and an altered capacity to direct Th cell differentiation.

In one embodiment, peripheral blood (PB) mononuclear cells which have been depleted of T, B, and NK cell populations are grown in culture according to the methods provided by the present invention. Monocytes cultured by the methods of the invention differentiate into APC of the invention, including unique subsets of dendritic cells. Like conventional monocyte-derived DC, designated herein as mDC1, the monocyte-derived dendritic cells of the present invention exhibit characteristic morphology and express high levels of dendritic cell markers on their surface, including MHC class I and class II molecules, CD11c, CD40, CD80, and CD86. Importantly, however, in contrast with conventional monocyte-derived dendritic cells, the dendritic cells of the invention lack cell surface expression of CDla (and thus are termed CD1a⁻ cells). Functionally, the novel dendritic cell subtype of the present invention differs from conventional dendritic cells by exhibiting a distinct cytokine production profile. Conventional dendritic cells express high levels of IL-12, a property which is significant in their role as antigen presenting cells. The dendritic cell subtypes of the present invention produce essentially no measurable IL-12 and produces increased level of IL-10 relative to the level of IL-10 produced by conventional dendritic cells. Notably, the lack of IL-12 and CDla expression by the monocyte-derived dendritic cells of the present invention does not affect their APC capacity, because they stimulate MLR to a similar degree as conventional monocyte-derived dendritic cells.

In contrast with conventional monocyte-derived dendritic cells which strongly favor Th1 differentiation, the unique monocyte-derived dendritic cells of the present invention favor differentiation of Th0/Th2 cells when co-cultured with purified human peripheral blood cells.

In addition, the monocyte-derived dendritic cells of the present invention exhibit a significantly higher transfection efficiency with plasmid DNA vectors than that of conventional monocyte-derived dendritic cells. The culture medium utilized is an important parameter in determining the differentiation pathway and phenotype of dendritic cells. In one embodiment, the present invention monocytes are cultured in a complex medium containing insulin, transferrin, linoleic acid, oleic acid and palmitic acid, with a combination of additives and growth factors which directs their differentiation, in vitro, ex vivo, or in vivo, along a heretofore undescribed pathway.

Definitions

Unless otherwise defined herein, all technical and scientific terms have the same meaning as commonly understood by one of ordinary skill in the art to which this invention nd pertains. Singleton et al. (1994) Dictionary of Microbiology and Molecular Biology, 2^{nd} edition, John Wiley and Sons (New York), and Kendrew (1994) The Encyclopedia of Molecular Biology, Blackwell Science Ltd. (London), provide one of skill with a general reference for many of the terms used in this invention. Paul (1998) Fundamental Immunology, 4^{th} edition, Raven Press (New York) and the references cited therein provide one of skill with a general overview of the ordinary meaning of many of the immunologically related terms used herein. Although any methods and materials similar or equivalent to those described herein can be used in the practice or testing of the present invention, preferred methods and materials are described. For the purposes of the present invention, the following terms are defined below.

An “antigen presenting cell” is any of a variety of cells capable of displaying, acquiring, or presenting at least one antigen or antigenic fragment on (or at) its cell surface.

A “dendritic cell” (DC) is an antigen presenting cell existing in vivo, in vitro, ex vivo, or in a host or subject, or which can be derived from a hematopoietic stem cell or a monocyte. Dendritic cells and their precursors can be isolated from a variety of lymphoid organs, e.g., spleen, lymph nodes, as well as from bone marrow and peripheral blood. The DC has a characteristic morphology with thin sheets (lamellipodia) extending in multiple directions away from the dendritic cell body. Typically, dendritic cells express high levels of MHC and costimulatory (e.g., B7-1 and B7-2) molecules. Dendritic cells can induce antigen specific differentiation of T cells in vitro, and are able to initiate primary T cell responses in vitro and in vivo.

Dendritic cells and T cells develop from hematopoietic stem cells along divergent “differentiation pathways.” A differentiation pathway describes a series of cellular transformations undergone by developing cells in a specific lineage. T cells differentiate from lymphopoietic precursors, whereas DC differentiate from precursors of the monocytemacrophage lineage.

“Cytokines” are protein or glycoprotein signaling molecules involved in the regulation of cellular proliferation and differentiation. Cytokines involved in differentiation and regulation of cells of the immune system include various structurally related or unrelated lymphokines (e.g., granulocyte-macrophage colony stimulating factor (GM-CSF), interferons (IFNs)) and interleukins (IL-1, IL-2, etc.)

A “polynucleotide sequence” is a nucleic acid (which is a polymer of nucleotides (A,C,T,U,G, etc. or naturally occurring or artificial nucleotide analogues) or a character string representing a nucleic acid, depending on context. Either the given nucleic acid or the complementary nucleic acid can be determined from any specified polynucleotide sequence.

An “amino acid sequence” is a polymer of amino acids (a protein, polypeptide, etc.) or a character string representing an amino acid polymer, depending on context. Either the given nucleic acid or the complementary nucleic acid can be determined from any specified polynucleotide sequence.

An “antigen” is a substance which can induce an immune response in a host or subject, such as a mammal. Such an antigenic substance is typically capable of eliciting the formation of antibodies in a host or subject or generating a specific population of lymphocytes reactive with that substance. Antigens are typically macromolecules (e.g., proteins, peptides, or fragments thereof; polysaccharides or fragments thereof) that are foreign to the host. A protein antigen or peptide antigen, or fragment thereof may be termed “antigenic protein” or “antigenic peptide,” respectively.” A fragment of an antigen is termed an “antigenic fragment.” An antigenic fragment has antigenic properties and can induce an immune response as described above.

An “immunogen” refers to a substance that is capable of provoking an immune response. Examples of immunogens include, e.g., antigens, autoantigens that play a role in induction of autoimmune diseases, and tumor-associated antigens expressed on cancer cells.

The term “immunoassay” includes an assay that uses an antibody or immunogen to bind or specifically bind an antigen. The immunoassay is typically characterized by the use of specific binding properties of a particular antibody to isolate, target, and /or quantify the antigen.

A vector is a composition or component for facilitating cell transduction by a selected nucleic acid, or expression of the nucleic acid in the cell. Vectors include, e.g., plasmids, cosmids, viruses, YACs, bacteria, poly-lysine, etc. An “expression vector” is a nucleic acid construct, generated recombinantly or synthetically, with a series of specific nucleic acid elements that permit transcription of a particular nucleic acid in a host cell. The expression vector can be part of a plasmid, virus, or nucleic acid fragment. The expression vector typically includes a nucleic acid to be transcribed operably linked to a promoter.

An “epitope” is that portion or fragment of an antigen, the conformation of which is recognized and bound by a T cell receptor or by an antibody.

A “target cell” is a cell which expresses an antigenic protein or peptide or fragment thereof on a MHC molecule on its surface. T cells recognize such antigenic peptides bound to MUC molecules killing the target cell, either directly by cell lysis or by releasing cytokines which recruit other immune effector cells to the site.

An “exogenous antigen” is an antigen not produced by a particular cell. For example, and exogenous antigen can be a protein or other polypeptide not produced by the cell that can be internalized and processed by antigen presenting cells for presentation on the cell surface. Alternatively, exogenous antigens (e.g., peptides) can be externally loaded onto MHC molecules for presentation to T cells.

An “exogenous” gene or “transgene” is a gene foreign (or heterologous) to the cell, or homologous to the cell, but in a position within the host cell nucleic acid in which the genetic element is not ordinarily found. Exogenous genes can be expressed to yield exogenous polypeptides. A “transgenic” organism is one which has a transgene introduced into its genome. Such an organism is either an animal or a plant.

“Transfection” refers to the process by which an exogenous DNA sequence is introduced into a eukaryotic host cell. Transfection (or transduction) can be achieved by any one of a number of means including electroporation, microinjection, gene gun delivery, retroviral infection, lipofection, superfection and the like. A “parental” cell, or organism, is an untransfected member of the host species giving rise to a transgenic cell, or organism.

The term “subject” or “host” as used herein includes, but is not limited to, an organism or animal; a mammal, including, e.g., a human, non-human primate (e.g., monkey), mouse, pig, cow, goat, rabbit, rat, guinea pig, hamster, horse, monkey, sheep, or other non-human mammal; a non-mammal, including, e.g., a non-mammalian vertebrate, such as a bird (e.g., a chicken or duck) or a fish, and a non-mammalian invertebrate.

The term “pharmaceutical composition” means a composition suitable for pharmaceutical use in a subject, including an animal or human. A pharmaceutical composition generally comprises an effective amount of an active agent and a pharmaceutically acceptable carrier.

The term “effective amount” means a dosage or amount sufficient to produce a desired result. The desired result may comprise an objective or subjective improvement in the recipient of the dosage or amount.

A “prophylactic treatment” is a treatment administered to a subject who does not display signs or symptoms of a disease, pathology, or medical disorder, or displays only early signs or symptoms of a disease, pathology, or disorder, such that treatment is administered for the purpose of diminishing, preventing, or decreasing the risk of developing the disease, pathology, or medical disorder. A prophylactic treatment functions as a preventative treatment against a disease or disorder. A “prophylactic activity” is an activity of an agent, such as a nucleic acid, vector, gene, polypeptide, protein, antigen or portion or fragment thereof, substance, or composition thereof that, when administered to a subject who does not display signs or symptoms of pathology, disease or disorder, or who displays only early signs or symptoms of pathology, disease, or disorder, diminishes, prevents, or decreases the risk of the subject developing a pathology, disease, or disorder. A “prophylactically useful” agent or compound (e.g., nucleic acid or polypeptide) refers to an agent or compound that is useful in diminishing, preventing, treating, or decreasing development of pathology, disease or disorder.

A “therapeutic treatment” is a treatment administered to a subject who displays symptoms or signs of pathology, disease, or disorder, in which treatment is administered to the subject for the purpose of diminishing or eliminating those signs or symptoms of pathology, disease, or disorder. A “therapeutic activity” is an activity of an agent, such as a nucleic acid, vector, gene, polypeptide, protein, antigen or portion or fragment thereof, substance, or composition thereof, that eliminates or diminishes signs or symptoms of pathology, disease or disorder, when administered to a subject suffering from such signs or symptoms. A “therapeutically useful” agent or compound (e.g., nucleic acid or polypeptide) indicates that an agent or compound is useful in diminishing, treating, or eliminating such signs or symptoms of a pathology, disease or disorder.

As used herein, an “antibody” refers to a protein comprising one or more polypeptides substantially or partially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (e.g., antibody) structural unit comprises a tetramer. Each tetramer is composed of two identical pairs of polypeptide chains, each pair having one “light” (about 25 kD) and one “heavy” chain (about 5070 kD). The N-terminus of each chain defines a variable region of about 100 to 110 or more amino acids primarily responsible for antigen recognition. The terms variable light chain (VL) and variable heavy chain (VH) refer to these light and heavy chains, respectively. Antibodies exist as intact immunoglobulins or as a number of well characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region thereby converting the (Fab′)2 dimer into an Fab′ monomer. The Fab′ monomer is essentially an Fab with part of the hinge region (see Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies. Antibodies include single chain antibodies, including single chain Fv (sFv) antibodies, in which a variable heavy and a variable light chain are joined together (directly or through a peptide linker) to form a continuous polypeptide.

An “antigen-binding fragment” of an antibody is a peptide or polypeptide fragment of the antibody which binds an antigen. An antigen-binding site is formed by those amino acids of the antibody which contribute to, are involved in, or affect the binding of the antigen. See Scott, T. A. and Mercer, E. I., CONCISE ENCYCLOPEDIA: BIOCHEMISTRY AND MOLECULAR BIOLOGY (de Gruyter, 3^{rd} e. 1997) (hereinafter “Scott, CONCISE ENCYCLOPEDIA”) and Watson, J. D. et al., RECOMBINANT DNA (2^{nd} ed. 1992) (hereinafter “Watson, RECOMBINANT DNA”), each of which is incorporated herein by reference in its entirety for all purposes.

A nucleic acid or polypeptide is “recombinant” when it is artificial or engineered, or derived from an artificial or engineered protein or nucleic acid. The term “recombinant” when used with reference e.g., to a cell, nucleotide, vector, or polypeptide typically indicates that the cell, nucleotide, or vector has been modified by the introduction of a heterologous (or foreign) nucleic acid or the alteration of a native nucleic acid, or that the polypeptide has been modified by the introduction of a heterologous amino acid, or that the cell is derived from a cell so modified. Recombinant cells express nucleic acid sequences (e.g., genes) that are not found in the native (non-recombinant) form of the cell or express native nucleic acid sequences (e.g., genes) that would be abnormally expressed under-expressed, or not expressed at all. The term “recombinant nucleic acid” (e.g., DNA or RNA) molecule means, for example, a nucleotide sequence that is not naturally occurring or is made by the combatant (for example, artificial combination) of at least two segments of sequence that are not typically included together, not typically associated with one another, or are otherwise typically separated from one another. A recombinant nucleic acid can comprise a nucleic acid molecule formed by the joining together or combination of nucleic acid segments from different sources and/or artificially synthesized. The term “recombinantly produced” refers to an artificial combination usually accomplished by either chemical synthesis means, recursive sequence recombination of nucleic acid segments or other diversity generation methods (such as, e.g., shuffling) of nucleotides, or manipulation of isolated segments of nucleic acids, e.g., by genetic engineering techniques known to those of ordinary skill in the art. “Recombinantly expressed” typically refers to techniques for the production of a recombinant nucleic acid in vitro and transfer of the recombinant nucleic acid into cells in vivo, in vitro, or ex vivo where it may be expressed or propagated. A “recombinant polypeptide” or “recombinant protein” usually refers to polypeptide or protein, respectively, that results from a cloned or recombinant gene or nucleic acid.

A “subsequence” or “fragment” is any portion of an entire sequence, up to and including the complete sequence.

The term “gene” broadly refers to any segment of DNA associated with a biological function. Genes include coding sequences and/or regulatory sequences required for their expression. Genes also include non-expressed DNA nucleic acid segments that, e.g., form recognition sequences for other proteins.

Generally, the nomenclature used hereafter and the laboratory procedures in cell culture, molecular genetics, molecular biology, nucleic acid chemistry, and protein chemistry described below are those well known and commonly employed by those of ordinary skill in the art. Standard techniques, such as described in Sambrook et al., Molecular Cloning—A Laboratory Manual (2nd Ed.), Vols. 1-3, Cold Spring Harbor Laboratory, Cold Spring Harbor, N.Y., 1989 (hereinafter “Sambrook”) and Current Protocols in Molecular Biology, F. M. Ausubel et al., eds., Current Protocols, a joint venture between Greene Publishing Associates, Inc. and John Wiley & Sons, Inc. (supplemented through 1999) (hereinafter “Ausubel”), are used for recombinant nucleic acid methods, nucleic acid synthesis, cell culture methods, and transgene incorporation, e.g., electroporation, injection, and lipofection. Generally, oligonucleotide synthesis and purification steps are performed according to specifications. The techniques and procedures are generally performed according to conventional methods in the art and various general references which are provided throughout this document. The procedures therein are believed to be well known to those of ordinary skill in the art and are provided for the convenience of the reader.

A variety of additional terms are defined or otherwise characterized herein.

Antigen Presentation

Pathogens and diseased cells, e.g., tumor, necrotic, or apoptotic cells, express a variety of antigens implicated in the cell-mediated immune response against the target cell. It is expected that one of ordinary skill in the art is familiar with the identity of many such antigens. T cells recognizing such epitopes are stimulated to proliferate in response to antigen presenting cells, such as dendritic cells, including the dendritic cells of the present invention, which display an antigen on a MHC molecule. Examples of antigens include tumor derived antigens, e.g., prostate specific antigen (PSA), colon cancer antigens (e.g., CEA), breast cancer antigens (e.g., HER-2), leukemia antigens, and melanoma antigens (e.g., MAGE-1, MART-1); antigens to lung, colorectal, brain, pancreatic cancers; antigens to renal cell carcinoma, lung, colorectal, pancreatic B-cell lymphoma, multiple myeloma, prostate carcinomas, sarcomas, and neuroblatomas; viral antigens, e.g., hepatitis B core and surface antigens (HBVc, HBVs), hepatitis A, B or C antigens, Epstein-Barr virus antigens, CMV antigens, human immunodeficiency virus (HIV) antigens, herpes virus antigens, and human papilloma virus (HPV) antigens; bacterial and mycobacterial antigens (e.g., for TB, leprosy, or the like); other pathogen derived antigens, e.g., Malarial antigens from Plasmodium sp.; or other cellular antigens, e.g., tyrosinase, trp-1. Many other antigen types are known and available, and can be presented by the DC of the invention.

Proteins or peptide fragments which are differentially expressed in cancers, such as those associated with melanoma (e.g., MART-1, gp100, TRP-1, TRP-2 or tyrosinase; see, e.g., Zhai et al.(1996) J Immunol. 156:700; Kawakami et al. (1994) J Exp Med. 180:347; and Topalian et al. (1994) 180:347; and Topalian et al. (1994) Proc Natl Acad Sci USA 91:9461) can be externally loaded onto or expressed in the DC of the invention for antigen presentation to T cells. Similarly, proteins associated with breast cancers (e.g., c-erb-2, bcl-1, bcl-2, and vasopressin related proteins; see, e.g., North et al. (1995) Breast Cancer Res Treat 34:229; Hellemans (1995) Br J Cancer 72:354; and Hurlimann et al. (1995) Virchows Arch 426:163; and other carcinomas (e.g., c-myc, int-2, hst-1, ras and p53 mutants, prostate-specific membrane antigen (PMSA) and papilloma virus protein L1, see Issing et el. (1993) Anticancer Res 13:2541; Tjoa et al. (1996) Prostate 28:65; Suzich et al. (1995) Proc Natl Acad Sci USA 92:11553; and Gjertsen (1995) Lancet 346:1399) are suitable antigens for external loading or expression. Choudhury et al. (1997) Blood 4:1133 describe the use of leukemic dendritic cells for autologous therapy against chronic myelogenous leukemias (CML); accordingly, it will be appreciated that leukemia antigens are beneficially presented by the DC of the invention. Other tumor antigens suitable for presentation include, but are not limited to, c-erb-β-2/HER2/neu, PEM/MUC-1, Int-2, Hst, BRCA-1, BRCA-2, EGFR, CEA, p53, ras RK, Myc, Myb, OB-1, OB-2, BCR/ABL, GIP, GSP, RET, ROS, FIS, SRC, TRC, WTI, DCC, Nfi, FAP, MEN-1, ERB-B1. See also Cell (1991) 64:235.

Antigens derived from pathogens, including viral, bacterial, intracellular and extracellular parasites are also suitable antigens for loading onto or expressing in the DC cells of the present invention. Numerous viral proteins are suitable for presentation by the DC of the invention, including those of papilloma viruses; HIV (e.g., Gag and Env antigens), see Gonda et al. (1992) in Kurstak et al. (eds.) Control of Virus Diseases, pp3-31; hepatitis, (e.g., HBs-Ag) among many others.

Mycobacteria, including species responsible for tuberculosis and leprosy, are the causative agents for a wide variety of disorders. In general, proteins expressed by mycobacteria and mycobacterially infected cells in the context of MHC are attractive targets for cell mediated therapies, because cells infected with the mycobacteria are killed by cytolysis, while antibody mediated therapies are often ineffective. Similarly, other infectious bacteria which also intracellularly infect cells, such as chlamydia, staphylococci, streptococci, pneumonococci, meningococci and conococci, klebsiella, proteus, serratia, pseudomonas, legionaella, diphtheria, salmonella, bacilli, cholera, tetanus, botulism, anthrax, plague, leptospirosis, rickettsial and Lyme disease bacteria, are suitable targets for cell mediated therapies. Antigens derived from the bacterial agents listed above as well as many others are suitable for display or presentation by the DC of the invention.

Antigens of cellular parasites, such as Malaria, are also appropriate for loading onto or expressing in the DC of the present invention. Malaria is caused by one of four species of Plasmodium: P. falciparum, P. vivax, P. knowlesi and P. malariae. Malaria is well studied, and a number of antigens suitable for cell mediated therapies are known.

In general, methods for peptide (or protein) loading for selected proteins and protein fragments onto dendritic cells are known in the art. See, e.g., WO 97/24447. In some embodiments, it is preferable to facilitate uptake of whole proteins by the DC, which process and express peptide fragments of the protein on their respective surfaces. In other cases, it is desirable simply to wash endogenous peptide fragments off of the surface of DC (e.g., in a mildly acidic or detergent containing wash) and to then load peptide fragments onto the surface of the cell. Many such applications are known in the art. For example, Tsai et al. (1997) J Immunol 158:1796 describe the loading of GP-100 tumor associated antigens onto DC. Alternatively, and for many applications, preferably, proteins or peptides comprising antigens can be expressed in DC or DC progenitors using recombinant DNA technology.

Peptide or protein antigens can also be delivered to APC and DC of the invention (e.g., mDC2) of the invention for display and presentation by commonly known pulsing methods. APC and DC of the invention of the invention can be pulsed with at least one peptide or protein antigen of interest ex vivo or in vitro. See, e.g., Nestle at al. (1998) Nature Medicine 4:328.

The genes encoding antigens of interest, and as described above, can be cloned and overexpressed in cells, including the DC of the invention or in DC progenitors, using standard techniques. General texts which describe molecular biological techniques useful herein, including the use of vectors, promoters and many other relevant topics related to, e.g., the cloning and expression of tumor or other cellular antigens, viral antigens, bacterial antigens, parasite antigens, or other antigens, include Berger and Kimmel, Guide to Molecular Cloning Techniques, Methods in Enzymology, Vol. 152, Academic Press, Inc., San Diego, Calif. (“Berger”); Sambrook et al., Molecular Cloning—A Laboratory Manual (2nd Ed.), Vol. 1-3, Cold Spring Harbor Laboratory, Cold Spring Harbor, N.Y., 1989 (“Sambrook”); and Current Protocols in Molecular Biology, F. M. Ausubel et al., eds., Current Protocols, a joint venture between Greene Publishing Associates, Inc. and John Wiley & Sons, Inc., (supplemented through 1999) (“Ausubel”).

Expression cassettes used to transfect cells preferably contain DNA sequences to initiate transcription and sequences to control the translation of any encoded antigenic protein or peptide sequence. These sequences are referred to as expression control sequences. Exemplary expression control sequences active in APC and dendritic cells of the invention are obtained from the SV-40 promoter (Science (1983) 222:5324), the CMV intermediate-early (I.E.) promoter (Proc Natl Acad Sci USA (1984) 81:659), and the metallothionein promoter (Nature (1982) 296:39). Pol III promoters, such as tRNAᵥₐ₁ (a house-keeping cellular gene promoter) and the adenovirus VA1 promoter (a strong viral promoter), are also desirable. Any of these, or other expression control sequences known in the art, can be used to regulate expression of polypeptides suitable for presentation by the DC of the present invention.

Polyadenylation or transcription terminator sequences from known mammalian genes are typically incorporated into the vector. Pol III termination sequences are outlined in Geiduschek (1988) Ann Rev Biochem 57:873. An example of a terminator sequence is the polyadenylation sequence from the bovine growth hormone gene. Sequences for accurate splicing of the transcript can also be included. An example of a splicing sequence is the VP1 intron from SV40 (Sprague et al. (1983) J Virol 45:773).

The cloning vector containing the expression control and/or transcription terminator sequences is cleaved using restriction enzymes and adjusted in size as necessary or desirable and ligated with nucleic acid coding for the target polypeptides by means well-known in the art.

Both naturally occuring, wild type and mutant, nucleic acids, as well as engineered or altered nucleic acids are favorably employed in the context of the present invention. One of skill will recognize many ways of generating alterations in a given nucleic acid sequence, such as a known cancer marker which encodes an antigen of interest. Such well-known methods include site-directed mutagenesis, PCR amplification using degenerate oligonucleotides, exposure of cells containing the nucleic acid to mutagenic agents or radiation, recursive sequence recombination and diversity generation methods of nucleotides (such as, e.g., DNA shuffling), chemical synthesis of a desired oligonucleotide (e.g., in conjunction with ligation and/or cloning to generate large nucleic acids) and other well-known techniques. See, e.g., Giliman and Smith (1979) Gene 8:81; Roberts et al. (1987) Nature 328:731; Stemmer (1994) Proc Natl Acad Sci U.S.A. 91:10747; Mullis et al. (1987) U.S. Pat. No. 4,683,202; PCR Protocols A Guide to Methods and Applications (Innis et al. eds) Academic Press Inc. San Diego, Calif. (1990) and Sambrook, Ausubel, and Berger (all supra).

To generate an altered nucleic acid (e.g., that encodes an antigenic peptide or protein, a cytokine or other costimulatory molecule, or that comprises a vector or vector component), any of a variety of diversity generating protocols, including nucleic acid shuffling protocols, are available and fully described in the art. The procedures can be used separately, and/or in combination to produce one or more variants of a nucleic acid or set of nucleic acids, wherein each nucleic acid encodes a peptide or protein (e.g., antigen) of interest, as well variants of encoded proteins. Individually and collectively, these procedures provide robust, widely applicable ways of generating diversified nucleic acids and sets of nucleic acids (including, e.g., nucleic acid libraries) useful, e.g., for the engineering or rapid evolution of nucleic acids, proteins, peptides, and pathways exhibiting new and/or improved characteristics (including, e.g., improved or enhanced immune responses), to be used in association with the dendritic cells of the present invention.

The following publications describe a variety of diversity generating procedures, including recursive sequence recombination procedures (also termed simply “recursive recombination), and/or methods for generating modified nucleic acid sequences for use in the procedures and methods of the present invention include the following publications and the references cited therein: Soong, N. W. et al. (2000) “Molecular Breeding of Viruses,” Nature Genetics 25:436-439; Stemmer, W. et al. (1999) “Molecular breeding of viruses for targeting and other clinical properties,” Tumor Targeting 4:1-4; Ness et al. (1999) “DNA Shuffling of subgenomic sequences of subtilisin,” Nature Biotechnology 17:893-896; Chang et al. (1999) “Evolution of a cytokine using DNA family shuffling,” Nature Biotechnology 17:793-797; Minshull and Stemmer (1999) “Protein evolution by molecular breeding,” Current Opinion in Chemical Biology 3:284-290; Christians et al. (1999) “Directed evolution of thymidine kinase for AZT phosphorylation using DNA family shuffling,” Nature Biotechnology 17:259-264; Crameri et al. (1998) “DNA shuffling of a family of genes from diverse species accelerates directed evolution,” Nature 391:288-291; Crameri et al. (1997) “Molecular evolution of an arsenate detoxification pathway by DNA shuffling,” Nature Biotechnology 15:436-438; Zhang et al. (1997) “Directed evolution of an effective fucosidase from a galactosidase by DNA shuffling and screening,” Proc. Nat'l Acad. Sci. USA 94:4504-4509; Patten et al. (1997) “Applications of DNA Shuffling to Pharmaceuticals and Vaccines,” Current Opinion in Biotechnology 8:724-733; Crameri et al. (1996) “Construction and evolution of antibody-phage libraries by DNA shuffling,” Nature Medicine 2:100-103; Crameri et al. (1996) “Improved green fluorescent protein by molecular evolution using DNA shuffling,” Nature Biotechnology 14:315-319; Gates et al. (1996) “Affinity selective isolation of ligands from peptide libraries through display on a lac repressor ‘headpiece dimer,’” J. Mol. Biol. 255:373-386; Stemmer (1996) “Sexual PCR and Assembly PCR” In: The Encyclopedia of Molecular Biology, VCH Publishers, New York. pp. 447-457; Crameri and Stemmer (1995) “Combinatorial multiple cassette mutagenesis creates all the permutations of mutant and wildtype cassettes,” BioTechniques 18:194-195; Stemmer et al. (1995) “Single-step assembly of a gene and entire plasmid form large numbers of oligodeoxy-ribonucleotides” Gene 164:49-53; Stemmer (1995) “The Evolution of Molecular Computation,” Science 270:1510; Stemmer (1995) “Searching Sequence Space,” Bio/Technology 13:549-553; Stemmer (1994) “Rapid evolution of a protein in vitro by DNA shuffling,” Nature 370:389-391; and Stemmer (1994) “DNA shuffling by random fragmentation and reassembly: In vitro recombination for molecular evolution,” Proc. Nat'l Acad. Sci. USA 91:10747-10751.

Additional details regarding DNA shuffling and other diversity generating methods can be found in the following U.S. patents, and international publications: U.S. Pat. No. 5,605,793 to Stemmer (Feb. 25, 1997), “Methods for In vitro Recombination;” U.S. Pat. No. 5,811,238 to Stemmer et al. (Sep. 22, 1998) “Methods for Generating Polynucleotides having Desired Characteristics by Iterative Selection and Recombination;” U.S. Pat. No. 5,830,721 to Stemmer et al. (Nov. 3, 1998), “DNA Mutagenesis by Random Fragmentation and Reassembly;” U.S. Pat. No. 5,834,252 to Stemmer (Nov. 10, 1998) “End-Complementary Polymerase Reaction;” U.S. Pat. No. 5,837,458 to Minshull (Nov. 17, 1998), “Methods and Compositions for Cellular and Metabolic Engineering;” WO 95/22625, Stemmer and Crameri, “Mutagenesis by Random Fragmentation and Reassembly;” WO 96/33207 by Stemmer and Lipschutz, “End Complementary Polymerase Chain Reaction;” WO 97/20078 by Stemmer and Crameri “Methods for Generating Polynucleotides having Desired Characteristics by Iterative Selection and Recombination;” WO 97/35966 by Minshull and Stemmer, “Methods and Compositions for Cellular and Metabolic Engineering;” WO 99/41402 by Punnonen et al. “Targeting of Genetic Vaccine Vectors;” WO 99/41383 by Punnonen et al., “Antigen Library Immunization;” WO 99/41369 by Punnonen et al., “Genetic Vaccine Vector Engineering;” WO 99/41368 by Punnonen et al., “Optimization of Immunomodulatory Properties of Genetic Vaccines;” WO 99/23107 by Stemmer et al., “Modification of Virus Tropism and Host Range by Viral Genome Shuffling;” WO 99/21979 by Apt et al., “Human Papillomavirus Vectors;” WO 98/31837 by Del Cardayre et al. “Evolution of Whole Cells and Organisms by Recursive Sequence Recombination;” WO 98/27230 by Patten and Stemmer, “Methods and Compositions for Polypeptide Engineering;” and WO 98/13487 by Stemmer et al., “Methods for Optimization of Gene Therapy by Recursive Sequence Shuffling and Selection;” WO 00/00632, “Methods for Generating Highly Diverse Libraries,” WO 00/09679, “Methods for Obtaining in vitro Recombined Polynucleotide Sequence Banks and Resulting Sequences,” WO 98/42832 by Arnold et al., “Recombination of Polynucleotide Sequences Using Random or Defined Primers,” WO 99/29902 by Arnold et al., “Method for Creating Polynucleotide and Polypeptide Sequences,” WO 98/41653 by Vind, “An in vitro Method for Construction of a DNA Library,” WO 98/41622 by Borchert et al., “Method for Constructing a Library Using DNA Shuffling,” and WO 98/42727 by Pati and Zarling, “Sequence Alterations using Homologous Recombination.”

As a review of the foregoing publications, patents, published foreign applications and U.S. patent applications reveals, diversity generation methods, such as shuffling (or recursive sequence recombination) of nucleic acids to provide new nucleic acids, e.g., antigens and/or vectors, with desired properties can be carried out by a number of established methods. Any of these methods can be adapted to the present invention to evolve new antigenic nucleic acids that can be used to transfect dendritic cells (e.g., mDC2) of the present invention such that at least one such nucleic acid is expressed and displayed or presented by the dendritic cell. In addition, any of these methods can be adapted to the present invention to evolve other components of expression vectors (e.g., promoter) that can be used for transfection of the DC (e.g., mDC2) of the invention.

Alternatively, any of these methods can be adapted to the present invention to evolve antigenic proteins or peptides that can be loaded into a dendritic cell of the invention such that at least one such antigenic peptide or protein is displayed or presented by the dendritic cell. Such dendritic cells of the invention displaying or presenting antigenic proteins or peptides are useful for inducing immune responses in subject in need of such treatment (as in vaccine or gene therapy applications). They are also useful in prophylactic and/or therapeutic methods for the treatment of diseases and disorders. Both the methods of making such dendritic cells and the cells produced by such methods are a feature of the invention.

Host cells, which can be bacterial or eukaryotic cells, are genetically engineered (i.e., transformed, transduced or transfected) with vectors suitable for expressing antigens which can be, for example, a cloning vector or an expression vector. The vector can be, for example, in the form of a plasmid, a viral particle, a phage, etc. The expression vector typically includes a promoter operably linked to the nucleic acid(s) encoding the antigen(s), and a polyadenylation sequence. In some embodiments, the expression vector is a part or portion of a plasmid construct. A plasmid construct may include, if desired, a marker(s) that can be selected, a signal component that allows the construct to exist as a single strand of nucleic acid, a bacterial origin of replication, a mammalian origin of replication (e.g., SV40), a multiple cloning site, and other components well known in the art.

The engineered host cells can be cultured in conventional nutrient media modified as appropriate for such activities as, for example, activating promoters or selecting transformants. The culture conditions, such as temperature, pH, and the like, are those previously used with the host cell selected for expression, and will be apparent to those skilled in the art and in the references cited herein, including, e.g., Freshney (1994) Culture of Animal Cells, a Manual of Basic Techniques, third edition, Wiley-Liss, New York and the references cited therein.

CD34⁺ stem cells transduced with a gene for an antigen of interest can be differentiated into dendritic cells in vitro. See, e.g., Reeves et al. (1996) Cancer Res 56:5672. Similarly, monocytes can be transfected with a gene for an antigen of interest and differentiated into DC by the methods of the invention.

Alternatively, the DC of the present invention can be directly transfected with a gene encoding an antigen of interest (or fragment thereof). The present invention provides subsets of dendritic cells which are amenable to transfection by a variety of means using conventional DNA vectors, e.g., electroporation of plasmid DNA, calcium phosphate precipitation, lipofection, gene gun delivery, delivery of naked DNA, and the like. Numerous techniques are available to one of skill in the art and are described in the references cited above, e.g., Ausubel, Sambrook, and Berger. Conventional DC have proven refractory to transfection with exogenous DNA sequences, regardless of the methods utilized. Typically, transfection rates are below 0.5%, making transfection of DC cells for therapeutic protocols a difficult, if not impossible task.

Limited success has been achieved using retroviral vectors to transfect hematopoietic stem cells (see, e.g., Hwu et al., PCT 97/29183 “METHODS AND COMPOSITIONS FOR TRANSFORMING DENDRITIC CELLS AND ACTIVATING T CELLS” published Aug. 14, 1997); however, the use of viral vectors is hampered by significant drawbacks. In particular, viral proteins expressed by the vector-infected DC cells activate virus-specific CTLs, resulting in lysis of the transfected DC. Plasmid vectors, in addition to avoiding the problems of viral-based vectors, offer several advantages over alternate vector technologies, such as excellent stability and ease of manufacturing and quality control.

The antigen presenting cells of the present invention (e.g., mDC2) permit the introduction of nucleic acids (e.g., DNA, RNA) into such cells (e.g., mDC2) with improved efficiency, thereby increasing their suitability for in vitro, and particularly for ex vivo and in vivo therapeutic and prophylactic applications, such as in immunotherapeutic applications (e.g., for cancer treatment) and genetic vaccine applications. Numerous methods suitable for introducing nucleic acids of interest, including those lacking retroviral sequences, into the dendritic cells of the invention are known in the art. For example, methods for introducing DNA sequences encoding antigenic proteins or peptides include Calcium phosphate precipitation, electroporation, microinjection, and gene gun delivery. Such methods are readily adaptable to a variety of DNA vectors, including expression vectors. Alternative methods include viral and retroviral infection, as well as methods involving lipid mediated uptake mechanisms such as lipofection, DOTAP supplemented lipofection, DOSPER supplemented lipofection and Superfection.

Furthermore, in some applications, e.g., ex vivo, in vitro, or in vivo applications for inducing an immune response, such as, e.g., prophylactic immunization (using vaccines or agents that promote an immune response), direct contact of a population of mDC2 cells with a nucleic acid (e.g., DNA) encoding an antigen of interest, wherein the sequence is operably linked to a promoter that controls expression of said sequence (e.g., a promoter that functions in a dendritic cell) in the absence of transfection-facilitating or transfection-enhancing agents (such as, e.g., viral particles, liposomal formulations, charged lipids, transfection-facilitating proteins, calcium phosphate-precipitating agents) is favorably employed. For example, it is well known to one of ordinary skill in the art that “naked” nucleic acids (e.g., naked DNA) can be used to transfect cells without transfection-facilitating calcium phosphate precipitating agents, liposomes, charged lipids or the like (see, e.g., U.S. Pat. Nos. 5,580,859 and 5,703,055.

A number of viral vectors suitable for in vitro, in vivo, or ex vivo transduction and expression are known and can be used for transduction, transfection, or transformation of the APC or mDC2 of the invention. Such vectors include retroviral vectors (see Miller (1992) Curr. Top. Microbiol. Immunol. 158:1-24; Salmons and Gunzburg (1993) Human Gene Therapy 4:129-141; Miller et al. (1994) Methods in Enzymology 217:581-599) and adeno-associated vectors (reviewed in Carter (1992) Curr. Opinion Biotech. 3:533-539; Muzcyzka (1992) Curr. Top. Microbiol. Immunol. 158:97-129). Other viral vectors that are used include adenoviral vectors, herpes viral vectors and Sindbis viral vectors, as generally described in, e.g., Jolly (1994) Cancer Gene Therapy 1:51-64; Latchman (1994) Molec. Biotechnol. 2:179-195; and Johanning et al. (1995) Nucl. Acids Res. 23:1495-1501. Such vectors may comprise a nucleic acid sequence encoding an antigen of interest that is to be displayed or presented on the APC or mDC2 of the invention, as well as a promoter operably linked to the nucleic acid(s) encoding the antigen(s), and a polyadenylation sequence, and, if desired other components as outlined above.

Several approaches for introducing nucleic acids into mDC2 cells in vivo, ex vivo and in vitro can be used. These include liposome based gene delivery (Debs and Zhu (1993) WO 93/24640 and U.S. Pat. No. 5,641,662; Mannino and Gould-Fogerite (1988) BioTechniques 6(7):682-691; Rose, U.S. Pat No. 5,279,833; Brigham (1991) WO 91/06309; and Felgner et al. (1987) Proc. Nat'l Acad. Sci. USA 84:7413-7414); Brigham et al. (1989) Am. J. Med. Sci. 298:278-281; Nabel et al. (1990) Science 249:1285-1288; Hazinski et al. (1991) Am. J. Resp. Cell Molec. Biol. 4:206-209; and Wang and Huang (1987) Proc. Nat'l Acad. Sci. USA 84:7851-7855); adenoviral vector mediated gene delivery, e.g., to treat cancer (see, e.g., Chen et al. (1994) Proc. Nat'l Acad. Sci. USA 91:3054-3057; Tong et al. (1996) Gynecol. Oncol. 61:175-179; Clayman et al. (1995) Cancer Res. 5:1-6; O'Malley et al. (1995) Cancer Res. 55:1080-1085; Hwang et al. (1995) Am. J. Respir. Cell Mol. Biol. 13:7-16; Haddada et al. (1995) Curr. Top. Microbiol. Immunol. 199 (Pt. 3):297-306; Addison et al. (1995) Proc. Nat'l Acad. Sci. USA 92:8522-8526; Colak et al. (1995) Brain Res. 691:76-82; Crystal (1995) Science 270:404-410; Elshami et al. (1996) Human Gene Ther. 7:141-148; Vincent et al. (1996) J. Neurosurg. 85:648-654), and many other diseases. Replication-defective retroviral vectors harboring therapeutic polynucleotide sequence as part of the retroviral genome have also been used, particularly with regard to simple MLV vectors. See, e.g., Miller et al. (1990) Mol. Cell. Biol. 10:4239 (1990); Kolberg (1992) J. NIH Res. 4:43, and Cornetta et al. (1991) Hum. Gene Ther. 2:215). Nucleic acid transport coupled to ligand-specific, cation-based transport systems (Wu and Wu (1988) J. Biol. Chem. 263:1462114624) have also been used. Naked DNA expression vectors have also been described (Nabel et al. (1990) Science 249:1285-1288); Wolff et al. (1990) Science, 247:1465-1468). In general, these approaches can be adapted to the invention by incorporating nucleic acids encoding an antigen or immunogenic peptide or protein to a disease or disorder, as described herein, into the appropriate vectors, and then using such vectors to transfect differentiated mDC2.

In addition to transfecting the dendritic cells of the invention with antigens or antigenic peptides of interest, it is sometimes desirable to introduce exogenous nucleic acids encoding non-antigenic proteins or peptides. For example, the efficacy of antigen presenting cells can be enhanced, or modulated, by transfecting nucleic acids encoding costimulatory molecules (e.g., CD28 binding proteins, CTLA-4 binding proteins, or other cell surface ligands and/or receptors) or cytokines.

Dendritic cells and DC progenitors which express or over-express transgenes encoding antigenic peptides, including polypeptides or proteins comprising an antigenic peptide, process and present the transgenic peptides on cell surface MHC molecules. This can be of particular use if naturally occurring sources of an antigenic peptide are scarce or difficult to manipulate, or if recovery is low.

Techniques are available in the art for stripping tumors of relevant antigens using a mild antigen wash (e.g., Zitvogel et al. (1996) J Exp Med 183:87). Antigens stripped in this manner can be externally loaded onto the DC of the present invention by incubating (or contacting) the cells with a source, such as culture medium containing, of the antigen according to well known procedures as described below. Similarly, bacterially, virally or parasitically infected cells are stripped of antigen and the resulting peptide mixture used to pulse load DC.

Commonly, proteins or peptides (including those which produce an antigenic or immune response) are made synthetically or recombinantly. Peptides and polypeptides to be loaded onto DC can be synthetically prepared in a wide variety of well-known ways. Polypeptides of relatively short size are typically synthesized in solution or on a solid support in accordance with conventional techniques. See, e.g., Merrifield (1963) J Am Chem Soc 85:2149. Various automatic synthesizers and sequencers are commercially available and can be used in accordance with known protocols. For example, see Stewart and Young (1984) Solid Phase Peptide Synthesis, 2^{nd} ed., Pierce Chemical Co. Polypeptides are also produced by recombinant expression of a nucleic acid encoding the polypeptide followed by purification using standard techniques.

DC are pulsed with these peptides at a concentration of about 0.0010-100 microliter/milliliter (μg/ml) at a cell density of about 1×10⁶ to 1×10⁷ per ml, often in the presence of β₂-microglobulin for roughly 2-6 hours, e.g., at about 20 ° C.-37 ° C. In some cases, it is beneficial to use a cationic lipid-protein complex (e.g., using the cationic lipid DOTAP complexed to the protein of interest) to aid in uptake of proteins for processing and presentation by dendritic cells. See, e.g., Nair et al. (1997) Int J Cancer 70:706. Carbohydrate antigens such as mucins are similarly loaded onto DC of the invention. The carbohydrate antigen is introduced into the DC as a moiety on a protein, or alternatively washed onto the DC. Such methods and variants known to those of skill in the art can be used to load peptides onto the DC of the invention.

Idiotypic antibodies are also appropriate antigens for the DC of the invention. Idiotypic antibodies are tumor antigens associated with a variety of conditions, e.g., lymphomas, leukemias, and the like, and are suitable for presentation by DC. For example, patients with non-Hodgkin's B-cell lymphoma who received an anti tumor vaccine of idiotypic Ig protein showed humoral, proliferative and CTL responses. See, e.g., Nelson et al. (1996) Blood 88:580. Other autoimmune disorders, such as multiple sclerosis, Rheumatoid arthritis, are also suitably treated by presenting idiotypic antibodies. Similarly, graft versus host and other transplantation rejection events can be treated by loading appropriate peptides onto the DC of the invention.

Isolation of Cells Using Selectable Markers

A variety of cells are used in the methods of the invention, including monocytes, T cells and dendritic cells. Each of these cell types is characterized by expression of particular markers on the surface of the cell, and lack of expression of other markers. For instance, in the mouse, some (but not all) dendritic cells express 33D1 (DC from spleen and Peyer's patch, but not skin or thymic medulla), NLDC145 (DC in skin and T-dependent regions of several lymphoid organs) and CD11c (CD11c also reacts with macrophage). T cells are positive for various markers depending on the particular subtype, most notably CD3, CD4 and CD8.

The expression of surface markers facilitates identification and purification of the various cells of the invention. These methods of identification and isolation include flow cytometry, column chromatography, panning with magnetic beads, western blots, radiography, electrophoresis, capillary electrophoresis, high performance liquid chromatography (HPLC), thin layer chromatography (TLC), hyperdiffusion chromatography, and the like, and various immunological methods, such as fluid or gel precipitin reactions, immunodiffusion (single or double), immunoelectrophoresis, radioimmunoassays (RIA), enzyme-linked immunosorbent assays (ELISA), immunofluorescent assays, and the like. For a review of immunological and immunoassay procedures in general, see Stites and Terr (eds.)(1991) Basic and Clinical Immunology, 7^{th} ed., and Paul, supra. For a discussion of how to make antibodies to selected antigens see, e.g., Coligan, supra; and Harlow and Lane (1989) Antibodies: A Laboratory Manual, Cold Spring Harbor Press, NY (“Harlow and Lane”).

Cell isolation or immunoassays for detection of cells, including the monocytes and dendritic cells of the invention, during cell purification can be performed in any of several configurations, including, e.g., those reviewed in Maggio (ed.)(1980) Enzyme Immunoassay, CRC Press, Boca Raton; Tjian (1985) “Practice and theory of enzyme immunoassays,” Laboratory Techniques in Biochemistry and Molecular Biology, Elsevier Science Publishers B.V., Amsterdam; Harlow and Lane, supra; Chan (ed.)(1987) Immunoassay: A Practical Guide, Academic Press, Orlando; and Price and Newman (eds.)(1991) Principles and Practice of Immunoassays, Stockton Press, NY, among others.

Most preferably, cells are isolated and characterized by flow cytometry methods such as fluorescence activated cell sorter (FACS) analysis. A wide variety of flow-cytometry methods are known. For a general overview of fluorescence activated flow cytometry see, for example, Abbas et al. (1991) Cellular and Molecular Immunology, W.B. Saunders Company; and Kuby (1992) Immunology, W.H. Freeman and Company, as well as other references cited above, e.g.,Coligan. Fluorescence activated cell scanning and sorting devices are available from e.g., Becton Dickinson, Coulter.

Labeling agents which can be used to label cellular antigens, including markers present on the surface of cells of the present invention, include, e.g., monoclonal antibodies, polyclonal antibodies, proteins, or other polymers, such as affinity matrices, carbohydrates, or lipids. Detection proceeds by any known method, such as immunoblotting, western blot analysis, tracking of radioactive or bioluminescent markers, capillary electrophoresis, or other methods which track a molecule based upon size, charge, or affinity. The particular label or detectable group used and the particular assay are not critical aspects of the invention. The detectable moiety can be any material having a detectable physical or chemical property. Such detectable labels have been well-developed in the field of gels, columns, solid substrates, cell cytometry and immunoassays, and, in general, any label useful in such methods can be applied to the present invention.

Thus, a label is any composition detectable by spectroscopic, photochemical, biochemical, immunochemical, electrical, optical or chemical means. Useful labels for detecting the cell populations, e.g., monocytes, dendritic cells, and T cells of the present invention include magnetic beads (e.g., Dynabeads™), fluorescent dyes (e.g., fluorescein isothiocyanate, Texas Red, rhodamine, and the like), radiolabels (e.g., ³H, ¹²⁵I, ³⁵S, ¹⁴C, or ³²P), enzymes (e.g., LacZ, CAT, horseradish peroxidase, alkaline phosphatase and others, commonly used as detectable enzymes, either as marker gene products or in an ELISA), nucleic acid intercalators (e.g., ethidium bromide) and colorimetric labels such as colloidal gold or colored glass or plastic (e.g., polystyrene, polypropylene, latex, etc.) beads.

The label is coupled directly or indirectly to the desired component of the assay according to methods well known in the art. As indicated above, a wide variety of labels are used, with the choice of label depending on the sensitivity required, ease of conjugation of the compound, stability requirements, available instrumentation, and disposal provisions. Non-radioactive labels are often attached by indirect means. Generally, a ligand molecule (e.g., biotin) is covalently bound to a polymer. The ligand then binds to an anti-ligand (e.g., streptavidin) molecule which is either inherently detectable or covalently bound to a signal system, such as a detectable enzyme, a fluorescent compound, or a chemiluminescent compound. A number of ligands and anti-ligands can be used. Where a ligand has a natural anti-ligand, for example, biotin, thyroxine, and cortisol, it can be used in conjunction with labeled, anti-ligands. Alternatively, any haptenic or antigenic compound can be used in combination with an antibody.

Labels can also be conjugated directly to signal generating compounds, e.g., by conjugation with an enzyme or fluorophore. Enzymes of interest as labels will primarily be hydrolases, particularly phosphatases, esterases and glycosidases, or oxidoreductases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems which are used, see, e.g., U.S. Pat. No. 4,391,904, which is incorporated herein by reference in its entirety for all purposes.

Means of detecting labels are well known to those of skill in the art. Thus for example, where the label is a radioactive label, means for detection include a scintillation counter or photographic film, as in autoradiography. Where the label is a fluorescent label, it is optionally detected by exciting the fluorochrome with the appropriate wavelength of light and detecting the resulting fluorescence, e.g., by microscopy, flow cytometry, visual inspection, via photographic film, by the use of electronic detectors such as charge coupled devices (CCD), photomultipliers, and the like. Similarly, enzymatic labels are detected by providing appropriate substrates for the enzyme and detecting the resulting reaction product. Finally, simple colorimetric labels are often detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of antibodies. In this case, cells e.g., the DC of the invention, are agglutinated by samples comprising the antibodies bound to the cell. In this format, none of the components need be labeled and the presence of the target antibody is detected by simple visual inspection.

Depending upon the assay, various components, including the antibody or anti-antibody, are typically bound to a solid surface. For instance, in a preferred embodiment, unwanted cells are panned out of cell culture using appropriate antibodies bound to a substrate over which the cells are passed. Many methods for immobilizing biomolecules to a variety of solid surfaces are known in the art. For example, the solid surface is optionally a membrane (e.g., nitrocellulose), a microtiter dish (e.g., PVC, polypropylene, or polystyrene), a test tube (glass or plastic), a dipstick, (e.g., glass, PVC, polypropylene, polystyrene, latex, and the like), a microcentrifuge tube, a flask, or a glass, silica, plastic, metallic or polymer bead. The desired component is optionally covalently bound, or noncovalently attached through nonspecific bonding. A wide variety of organic and inorganic polymers, both natural and synthetic are optionally employed as the material for the solid surface. Illustrative polymers include polyethylene, polypropylene, poly(4-methylbuten), polystyrene, polymethacrylate, poly(ethylene terephthalate), rayon, nylon, poly(vinyl butyrate), polyvinylidene difluoride (PVDF), silicones, polyformaldehyde, cellulose, cellulose acetate, nitrocellulose, and the like. Other materials which are appropriate depending on the assay include paper, glasses, ceramics, metals, metalloids, semiconductive materials, cements and the like. In addition, substances that form gels, such as proteins (e.g., gelatins), lipopolysaccharides, silicates, agarose and polyacrylamides can be used. Polymers which form several aqueous phases, such as dextrans, polyalkylene glycols or surfactants, such as phospholipids, long chain (12-24 carbon atoms) alkyl ammonium salts and the like are also suitable.

Isolation of Dendritic Cell Precursors

Dendritic cells are bone marrow-derived cells present at low density in the spleen and lymph nodes as well as in peripheral blood, where they are present at low numbers, <1%. They are characterized by their large size and unusual shape, a deficiency of macrophage and lymphocyte specific markers (e.g., Fc receptors), expression of high levels of Major Histocompatibility (MHC) Class II and costimulatory molecules, and potent T cell stimulatory activity.

Dendritic cell progenitors can be isolated from bone marrow and peripheral blood by flow cytometry as described above and below. Differentiation of mature dendritic cells from the monocyte lineage can be stimulated in vivo and in vitro with appropriate cytokine treatment, including culture in the presence of Granulocyte-Macrophage Colony-Stimulating Factor (GM-CSF), Tumor Necrosis Factor-α (TNF-α), and the CD40 ligand (CD40L). Typically, CD34+peripheral blood monocytes cultured in the presence of GM-CSF and IL-4 as well as cytokines derived from activated monocytes, give rise to cells with characteristic DC morphology that express CD1a (i.e., are CD1a⁺), designated herein as mDC1, alternatively referred to as “conventional” dendritic cells.

Additional details regarding methods for recovery and differentiation of dendritic cells are provided, e.g., in WO 98/05795 “ENRICHMENT OF DENDRITIC CELLS FROM BLOOD” by Crawford et al., published Feb. 12, 1998; WO 98/53048 “METHODS AND COMPOSITIONS FOR MAKING DENDRITIC CELLS FROM EXPANDED POPULATIONS OF MONOCYTES AND FOR ACTIVATING t CELLS” by Nelson et al., published Nov. 26, 1998; WO 97/29182 “Method and compositions for obtaining mature dendritic cells” BY Steinman et al., published Aug. 14, 1997; and U.S. Pat. No. 5,994,126 “METHOD FOR IN VITRO PROLIFERATION OF DENDRITIC CELL PRECURSORS AND THEIR USE TO PRODUCE IMMUOGENS” to Steinman et al., issued Nov. 30, 1999.

As described in greater detail below, the present invention provides culture conditions for generating DC subtypes that lack cell surface expression of CDla (i.e., thus are CD1a⁻), designated herein as “mDC2.” The mDC1and mDC2 subsets are further distinguished on the basis of their respective cytokine production profiles, and their different abilities to bias differentiation of T cells to the Th1 (T helper 1) cells or Th0/Th2, respectively. Specifically, mDC2 show substantially lower production of IL-12 than do mDC1. mDC2 also show an increased production of IL-10 as compared to the amount of IL-10 produced by mDC1. Furthermore, while mDC1 strongly bias the differentiation of T cells to Th 1 cells, mDC2 bias the T cell differentiation along the Th0/Th2 pathway, favoring the differentiation of T cells to Th2 and Th0. Furthermore, the mDC2 subtype demonstrates improved transfection efficiency relative to conventional mDC1 cells, enhancing their utility in numerous therapeutic and experimental applications, as will become clear upon review of the forthcoming discussion.

Dendritic cell (DC) progenitors can be isolated from a variety of lymphoid and non-lymphoid tissues. While spleen, lymph node and bone marrow are all suitable tissues, and can be used by preference in experimental animals, peripheral blood provides a convenient, minimally-invasive source of human dendritic cells progenitors useful for therapeutic applications. As is discussed further below, in applications involving, e.g., human subjects, it is generally desirable to obtain such progenitors from the same subject as targeted for subsequent 2-5 intervention utilizing the mature dendritic cells of the invention. Peripheral blood mononuclear cells can be isolated by centrifugal elutriation or density gradient centrifugation e.g., following leukapheresis or standard buffy coat preparation. Additional details relating to these and other techniques relevant to one skilled in the art for the preparation and manipulation of immunologically active cells can be found in e.g., Coligan et al. (eds.) (1991) Current Protocols in Immunology, and Supplements, John Wiley and Sons, Inc. (New York).

In preferred embodiments, monocytes are differentiated into dendritic cells. One of skill will appreciate that many therapeutic applications are improved by administering autologous cells to a subject (such as a patient), i.e., cells which were originally isolated from the subject, or which are derived from a subject by culturing isolated cells. These autologous cells are less likely to cause immune complications (e.g., host versus graft reactions) upon reintroduction or administration into the subject.

In preferred embodiments density gradient centrifugation (using e.g., Histopaque, Ficoll, etc.) is employed prior to negative depletion of T, B and NK cells by any of a variety of techniques well known in the art, (e.g., antibody conjugated magnetic beads, panning, complement mediated lysis) mononuclear cells are recovered and plated into appropriate culture medium. For example, mononuclear cells recovered after Histopaque density gradient centrifugation, are labeled with monoclonal antibodies specific for CD3, CD16, CD19 and CD56. Labeled cells are then incubated with mouse-Ab reactive immunomagnetic beads (e.g., Dynabeads™, Dynal, Oslo, Norway) for 30 minutes at 4° C. with gentle rotation, and positive cells are removed with a magnet. Monocytes can also be obtained from peripheral blood by positive selection using, for example, adherence to plastic or monocyte-specific monoclonal antibodies combined with panning, immunomagnetic beads or flow cytometry. After washing in isotonic saline, e.g., phosphate-buffered saline (PBS) with 2% fetal bovine serum (FBS), purified monocytes are collected and resuspended in culture medium at a concentration of 1×10⁶/ml. Alternatively, bone marrow aspiration from iliac crests (or other sites) can be performed, and mononuclear cells purified as described above.

Methods for Producing Dendritic Cells of the Invention

The present invention provides methods and culture conditions for producing and differentiating APC and DC with unique characteristics and properties, including distinctive cytokine production profiles, CD1a expression profiles, capacities to support Th cell differentiation, and/or transfection efficiency characteristics. Such methods are useful for producing the novel APC and DC of the invention, such as mDC2, which can be subsequently used in methods for treating diseases, as adjuvants, in vaccine applications, etc.

A population of conventional dendritic cells is produced by culturing a population of monocytes in RPMI medium in the presence of IL-4 and GM-CSF, as described by Sallusto and Lanzavechia (1994) “Efficient presentation of soluble antigen by cultured human dendritic cells is maintained by granulocyte/macrophage colony-stimulating factor plus interleukin 4 and downregulated by tumor necrosis factor alpha,” J Exp Med 179:1109. Under such conditions, the monocytes differentiate into conventional DC, which express CD1a, and other cell surface markers (as noted above). Further, conventional DC generated in the presence of IL-4 and GM-CSF in RPMI medium produce high levels of IL-12 (Macatonia et al. (1995) “Dendritic cells produce IL-12 and direct the development of TH1 cells from naive CD4⁺ T cells,” J Immunol 154:5071; Koch et al. (1996) “High level IL-12 production by murine dendritic cells: upregulation via MHC class II and CD40 molecules and downregulation by IL-4 and IL-10.” J Exp Med 184:741). The components of standard RPMI medium used for differentiation of monocytes to conventional DC are shown in Gibco BRL Life Technologies Products & Reference Guide 2000-2001, p. 1-62 1640 (see RPMI 1640 media, Catalog Nos. shown on p. 15 62, preferably 11875) and Moore, G. E., Gerner, R. E. and Franklin (1967) A.M.A. 199:519), each of which is incorporated herein by reference in its entirety for all purposes. RPMI medium comprises an enriched formulation for mammalian cells. Both IL-6 and IL-10 inhibit production of IL-12: however, cells cultured in the presence of IL-6 or IL-10 remain CD 14⁺, indicating that these cytokines also prevent DC differentiation.

The present invention identifies culture conditions and additives that induce differentiation of unique subtypes or subsets of DC that are phenotypically and functionally different from conventional DC produced in RPMI. In one embodiment, the mDC2 of the invention are produced by culturing a population of mononuclear cells or monocytes with IL-4, GM-CSF, and a culture medium comprising Iscove's Modified Dulbecco's Medium (IMDM) (as described in the Gibco BRL Life Technologies Products & Reference Guide 2000-2001, http://www.lifetech.com, Gibco BRL Life Technologies Rockville, Md. (see, e.g., the IMDM a media described in Gibco BRL Life Technologies Products & Reference Guide 2000-2001, p. 1-52, Catalog Nos. 12200, 12440, 31980, and preferably 21056), which is incorporated herein by reference in its entirety for all purposes. Other growth factors and additives, such as insulin, transferrin, and lipids or fatty acids (e.g., C₁₆-C₁₈ fatty acids, and isomers, derivatives, and analogs thereof) can also be used to supplement IMIDM to generate mDC2 possessing the phenotypic and/or functional characteristics described herein. For examples of C₁₆-C₁₈ fatty acids, and isomers, derivatives, and analogs thereof, see Voet, Voet, and Pratt, Fundamentals of Biochemistry (John Wiley & Sons, Inc. 1999), which is incorporated by reference herein in its entirety for all purposes.

In another embodiment, the invention provides a method of producing a differentiated APC (or mDC2) of the invention that comprises culturing a population of mononuclear cells or monoctyes with IMDM medium (e.g., Gibco BRL Life Technologies Products & Reference Guide 2000-2001, p. 1-52, Catalog Nos. 12200, 12440, 31980, and preferably 21056) supplemented with additives insulin, transferrin, linoleic acid, oleic acid, and palmitic acid, thereby producing differentiated APC (or mDC2) of the present invention. The amount of each such additive can be varied, but is an amount sufficient to induce or assist in differentiation of the monocyte. It is preferable to employ the additives within biologically relevant ranges.

Typically, in methods for producing differentiated APC and DC of the invention (e.g., mDC2) of the present invention, the culture medium comprises IMDM (e.g., Gibco BRL Life Technologies Products & Reference Guide 2000-2001, p. 1-52, Catalog Nos. 12200, 12440, 31980, and preferably 21056) with the following additives: insulin (Sigma; St. Louis, Mo.), from about 0.25-100, 1-50, 1-25, 1-15, 1-10, or 2-10 μg/ml; human transferrin (Boehringer Mannheim, Mannheim, Germany), from about 0.25-100, 1-100, 5-100, 5-50, or 5-30 microgram/milliliter (ug/ml); linoleic acid (Sigma), from about 0.25-100, 1-50, 1-25, 1-15, or 1-10 μg/ml; oleic acid (Sigma), from about 0.25-100, 1-50, 1-25, 1-15, or 1-10 μg/ml; palmitic acid (Sigma), from about 0.25-100, 1-50, 1-25, 1-15, or 1-10 μg/ml; and, optionally, also including one or more of: bovine serum albumin (BSA) (Sigma), from about 0.01-10% or 0.10.5% (w/v); 2-amino ethanol (Sigma), from about 0.25-10, 0.25-5, or 1-5 milligrams/liter (mg/L); fetal bovine serum (FBS) (Hyclone, Logan, UT), from about 0.5-50%, 1-20%, or 5-15%; and glutamine, from about 0.25-20, 0.25-10, 0.25-5, or 1-5 milliMolar (mM).

In yet another embodiment, the invention provides a method for producing a differentiated APC or mDC2 of the invention which comprises culturing a population of mononuclear cells or monocytes with IL-4, GM-CSF and a culture medium comprising IMDM (see, e.g., the IMDM media described in Gibco BRL Life Technologies Products & Reference Guide 2000-2001, p. 1-52, Catalog Nos. 12200, 12440, 31980, and preferably 21056) supplemented with insulin, 5 μg/ml; human transferrin, 20 μg/ml; linoleic acid 2 μg/ml; oleic acid, 2 μg/ml; and palmitic acid 2 μg/ml. In addition, the medium may be supplemented with from about 10-100 Units/milliliter (U/ml) (preferably about 50 U/ml) penicillin; from about 20-500 μg/ml (preferably about 100 μg/ml) streptomycin; from about 0.1-10% (weight/volume (w/v) bovine serum albumin (BSA) (preferably, 0.25% BSA (w/v)); from about 0.1-10 ug/ml 2amino ethanol (preferably, 1.8 ug/ml); and from about 1-40% fetal bovine serum (preferably 10% fetal bovine serum); and from about 0.5-10 mM glutamime (preferably 2 mM glutamine). In such method, sufficient time and culture conditions are permitted to allow for differentiation of the monocytes into the differentiated APC or mDC2 of the invention (as described below in greater detail and in the Examples below).

In a preferred embodiment, the invention provides a method for producing a differentiated APC or mDC2 of the invention which comprises culturing a population of mononuclear cells or monocytes in IL-4, GM-CSF, and “Yssel's medium” for a time and under culture conditions, as described below in greater detail and in the Examples below, sufficient to allow the monocytes to differentiate into the differentiated APC or mDC2 of the invention. Yssel's medium, which is described in Yssel et al. (1984) “Serum-free medium for generation and propagation of functional human cytotoxic and helper T cell clones,” J Immunol Methods 72(1):219, which is incorporated herein by reference in its entirety for all purposes, contains IMDM (see Gibco BRL Life Technologies Products & Reference Guide 2000-2001, p. 1-52, Catalog Nos. 12200, 12440, 31980, and preferably 21056) supplemented with insulin, 5 μg/ml; human transferrin, 20 μg/ml; linoleic acid 2 μg/ml; oleic acid, 2 μg/ml; palmitic acid 2 μg/ml;

bovine serum albumin (BSA), 0.25% (w/v); and 2-amino ethanol, 1.8 μg/ml), as described by Yssel, supra. Preferably, the IMDM is that designated by Catalog No. 21056 in Gibco BRL Life Technologies Products & Reference Guide 2000-2001, p. 1-52. In such method, sufficient time and culture conditions are permitted to allow for differentiation of the monocytes into the differentiated APC or mDC2 of the invention (as described below in greater detail and in the Examples below).

In all of the above-described methods for producing APC of the invention, the culture medium usually contains from about 10-100 Units/milliliter (U/ml) (preferably about 50 U/ml) penicillin; from about 20-500 μg/ml (preferably about 100 μg/ml) streptomycin; from about 1-40% fetal bovine serum (preferably 10% fetal bovine serum); and from about 0.5-10 mM glutamine (preferably 2 mM glutamine).

As noted above, other lipids or fatty acids (e.g., C₁₆-C₁₈ fatty acids, and isomers, derivatives, and analogs thereof) can be used to supplement IMDM to generate APC or mDC2 possessing the phenotypic and/or functional characteristics described herein. Preferably, a lipid that relates in chemical function or structure to one (or more) particular lipid(s) specified in the methods above can be substituted for the particular lipid. For example, alpha- or gamma-linoleic acid may be substituted in similar amount for linoleic acid, and palmitoleic acid may be substituted for palmitic acid. One of ordinary skill in the art will readily understand common lipids or fatty acids that can be substituted for the lipids or fatty acids specified in the methods above. For additional examples of C₁₆-C₁₈ fatty acids, and isomers, derivatives, and analogs thereof, including analogs, derivatives, and isomers of oleic acid, linoleic acid, and palmitic acid, see Voet, Voet, and Pratt, Fundamentals of Biochemistry (John Wiley & Sons, Inc. 1999), which is incorporated by reference herein in its entirety for all purposes.

In an alternative embodiment, the invention provides methods for producing differentiated APC or mDC2 of the invention, as defined by any of the methods described above, except that Dulbecco's Modified Eagle Medium (DMEM) is substituted for IMDM. The components of various DMEM media are described in the Gibco BRL Life Technologies Products & Reference Guide 2000-2001 (www.lifetech.com), p.1-45 (see, e.g., Catalog No. 11965).

Variations in the composition of the culture medium, e.g., glucose concentration, amino acid or nucleotide content, alcohol (e.g., ethanol) content, lipid content, vitamin supplementation, antibiotic supplementation, etc., can be made without significantly affecting the production of the dendritic cells of the invention. For example, a component exhibiting the same or similar properties as a component described in, e.g., Yssel's medium, can be substituted for the Yssel medium component.

For example, in one embodiment, a lipid relating to or derived from one or more of linoleic acid, oleic acid, or palmitic acid, such as a derivative, analog, or lipid exhibiting the same or comparable properties to linoleic acid, oleic acid, or palmitic acid, respectively, can be used in place of the respective lipid. Such a lipid may relate chemically or structurally to a lipid specified in Yssel et al., supra. Similarly, alternative lipid constituents and/or concentrations can be utilized. Suitable variants and alternatives medium compositions can be readily ascertained experimentally by one of skill in the art. In some cases, variations in the medium composition results in a phenotype intermediate between the mDC1 and mDC2 dendritic cell subtypes as described in further detail in the examples below. Mononuclear cells isolated as described above are introduced into the described culture medium, and typically maintained at or about 37 ° C., 5% CO₂, in a humidified atmosphere until they acquire a mature differentiated dendritic cell phenotype as assessed by cell surface markers and morphology (see, e.g., Example 1). During the course of the incubation, partially differentiated cells committed to a monocyte-dendritic cell differentiation pathway are also present in a mixed culture comprising dendritic cell progenitors and/or differentiated dendritic cells. It will be appreciated that, if desired, either during or following differentiation, the dendritic cells of the invention can be enriched, e.g., purified, from the population by flow cytometry as described above.

Antigen-presenting Cells of the Invention

The present invention provides mononuclear cell- or monocyte-derived APC and DC subsets (or subtypes) exhibiting phenotypically and functionally novel properties, features, and characteristics. For clarity and to distinguish these novel dendritic cells from conventional DC, DC of the present invention exhibiting the characteristics, features and properties described herein are termed “mCD2,” or dendritic cells (DC) of the present invention. Conventional DC exhibiting commonly known characteristics, features and properties are termed “mDC1” or conventional DC.

In one aspect, the invention provides a differentiated antigen presenting cell (APC), which differentiated APC does not express CDla cell surface marker. The differentiated APC may comprise a monocyte-derived CD1a⁻ dendritic cell. In some such aspects, the monocyte-derived CD1a⁻ dendritic cell substantially lacks IL-12 production, induces or promotes differentiation of T cells to Th0/Th2 subtypes, and/or is produced by culturing a population of monocytes in interleukin-4 (IL-4), granulocyte macrophage colony stimulating factor (GM-CSF), and a culture medium comprising Iscove's Modified Dulbecco's Medium (IMDM) supplemented with insulin, transferrin, linoleic acid, oleic acid and palmitic acid. Some such APC are produced using Yssel's medium. In some instances, the monocyte-derived CD1a⁻ dendritic cell has substantially increased IL-10 production as compared to a dendritic cell produced by culturing a population of peripheral blood or bone marrow mononuclear cells in IL-4, GM-CSF, and a culture medium comprising RPMI. In certain aspects, the monocyte-derived CD1a⁻ dendritic cell comprises an mDC2 and/or has a transfection efficiency greater than that of a dendritic cell produced by culturing a population of monocytes in IL-4, GM-CSF, and a culture medium comprising RPMI.

As described in greater detail above and below, in one aspect, the mDC2 of the present invention were produced by culturing a population of isolated monocytes in a unique culture medium comprising IMDM supplemented with insulin, transferrin, and lipids (such as oleic acid, palmitic acid, and linoleic acid, or chemical or structural derivatives, analogs, or isomers thereof). The culture medium may also be supplemented with IL-4 and GM-CSF. In another embodiment, the mDC2 of the invention were generated by culturing a population of isolated monocyte cells in Yssel's medium (described above and in Yssel, supra). Additionally, mDC2 can be produced by culturing a population of isolated monocyte cells in other media and conditions as described above in “Generation of Dendritic Cells.”

Like conventional monocyte-derived DC, mDC2 of the present invention express high levels of MHC molecules and costimulatory molecules, CD11c, CD40, CD80, and CD86. However, in contrast with mDC1 cells, the novel mDC2 of the present invention have an unusual phenotype in that they lack cell surface expression of CD1a (i.e., they are CD1a⁻), while expressing high levels of the other DC-associated antigens. This suggests an association between cytokine production profile and CDla expression in DC.

The mDC2 of the present invention are further distinguished from mDC1 by their cytokine production profile. MDC2 secrete increased levels of IL-10 compared with mDC1. Additionally, mDC2 produce no IL-12 upon activation with LPS plus IFN-γ or anti-CD40 mAbs, LPS plus IFN-γ, whereas conventional mCD1 cells produce high levels of IL-12 when activated under identical culture conditions.

The mDC2 of the present invention are also distinguished functionally from mDC1 in their direction of the differentiation of T helper (Th) cell subsets. While mDC1 strongly favor Th1 differentiation, mDC2 direct and bias differentiation toward the Th0/Th2 phenotype when co-cultured with purified human peripheral blood cells. The reduced IL-12 production of mDC2 is associated with the improved capacity of mDC2, as compared to conventional mDC1, to direct Th0/Th2 cell differentiation. MDC1 and mDC2 direct the differentiation of Th subsets with different cytokine production profiles.

mDC2 of the present invention were similar to mDC1 in their ability to induce potent proliferation of allogeneic T cells. No significant difference in the capacity of mDC1 and mDC2 to induce MLR was observed, irrespective of whether the cells expressed CD83. MDC2 can act as potent antigen-presenting cells.

The mechanisms initiating Th2 cell differentiation have been intensely investigated, because professional APCs, such as DC, are known to produce large quantities of IL-12, the most potent cytokine directing Th1 response. The underlying mechanisms mediating Th2 cytokines IL-4 and IL-13 dominate in certain disease situations, such as allergy resulting in increased IgE production (Punnonen et al. (1993) Proc Natl Acad Sci USA 90:3730; Punnonen et al (1998), in Allergy and Allergic Diseases: The New Mechanisms and Therapeutics 0. Denburg ed. Humana Press, Totowa, p.13). IL-4 is well known to efficiently direct Th2 responses, but no IL-4 production has been demonstrated by professional APCs. NK1.1⁺ T cells, a numerically minor T cell subset, have been shown to produce high levels of IL-4 and are likely to contribute to the initiation of Th2 response (Yoshimoto et al. (1995) Science 270:1845). However, they are not likely to be the only explanation, because APC typically secrete high levels of IL-12. It was recently shown that plasmacytoid cell-derived DC produce low levels of IL-12 and direct Th2 differentiation, whereas monocyte-derived DC produce high levels of IL-12 and skew T cell differentiation towards Th1 (Rissoan et al. (1999) Science 283:1183), indicating that APCs do differ in their capacity to produce cytokines. Importantly, however, two different cell populations were used as the starting material to generate these subsets, and it remained unclear whether one population has the capacity to differentiate DC subsets with different cytokine production profiles and capacities to mediate Th cell differentiation (Rissoan, supra; Bottomly (1999) Science 283:1124). With results described herein and the mDC2 of the present invention demonstrate that PB monoctyes can differentiate into at least two different subsets that differ from each other in cytokine synthesis profile, surface marker expression and capacity to direct Th differentiation.

mDC2 can be matured into CD83⁺ DC cells in the presence of anti-CD40 mAbs, followed by activation with LPS plus IFN-γ, while remaining CD1a⁻ and lacking IL-12 production even upon maturation. Even though they produce little or no IL-12 and do not express CD1a⁻, mDC2 still function with an antigen presenting cell (APC) capacity similar to that of mDC1 (as shown by the fact that mDC2 stimulated mixed lymphocyte reactions (MLR) to the similar degree as mDC1). This suggests there are similarities in the APC functions of these two cell populations.

In contrast to mDC1, mDC2 do not mature into CD83⁺ DC in the presence of LPS plus IFN-γ, indicating the signaling requirements for maturation between these two DC subsets are not identical. In addition, because mCD1 molecules can act as efficient lipid antigen-10 presenting molecules (Beckman et al. (1994) Nature 372:691; Sugita et al. (1999) Immunity 11:743), the fact that mDC2 remain CD1a⁻ upon maturation further supports the belief that the mDC2 subset is phenotypically and functionally distinct from the mDC1 subset.

The exact mechanisms that direct differentiation of mDC2 require further study, but it appears that DC differentiation is dependent on a delicate balance of growth factors in the microenvironment of the cells. PGE₂ has been previously shown to inhibit IL-12 production by monocytes cultured in the presence of IL-4 and GM-CSF, which was associated with increased capacity of these cells to direct Th2 differentiation (Kalinski et al. (1997) J. Immunol 159:28). However, APC cultured in the presence of PGE₂ retain characteristics of monocytes/macrophages, including expression of CD14 (see Kalinski et al., supra). In addition, PGE₂ supports maturation of CD1a⁺ DC (Kalinski et al. (1998) J. Immunol. 161:2804), whereas mDC2 remain CD1a⁻ upon maturation to CD83⁺ cells, further indicating that mDC2 are distinct from DC cultured in the presence of PGE₂. Yssel's medium, which provided the necessary signals to support mDC2 differentiation, is based on IMDM and additionally contains insulin, transferrin, linoleic acid, oleic acid and palmitic acid, all of which have been shown to affect the function of lymphoid cells in vitro and/or in vivo (28-32). IMDM also contains higher levels of glucose and several vitamins than RPMI, and glucose has previously been shown to enhance IL-6 and TNF-γ (gamma) production by monocytes (33). However, no single component of Yssel's medium was able to support mDC2 differentiation when added to RPMI, suggesting synergistic effects by the components of Yssel's medium in inducing mDC2 differentiation. Further studies are required to identify the relative contribution of each component and to investigate whether analogous conditions are present in vivo; for example, at the sites of inflammation. Nevertheless, these data support the conclusion that mDC2 differentiation is dependent on a delicate balance of multiple growth factors present in the microenvironment of the cells.

mDC2 produced increased levels of IL-10 as compared to mDC1 following activation with LPS plus IFN-γ, suggesting that endogenously produced IL-10 may play a role in regulating the function of mDC2. Recombinant IL-10 also inhibited IL-12 production by dendritic cells, which is consistent with previous studies indicating that IL-10 prevents cytokine synthesis and the accessory cell function of monocytes and DC (15, 42, 43). However, when recombinant IL-10 was added to DC cultured in the presence of RPMI, the cells also remained CD14⁺, strongly suggesting that IL-10 is not the underlying mechanism mediating mDC2 differentiation. Similar to IL-10, IL-6 inhibited IL-12 production by DC activated with LPS+IFN-gamma. Again, however, IL-6 also prevented DC differentiation as determined by the expression of CD14 on the cultured cells, which is in line with a previous study demonstrating that IL-6 inhibits the capacity of BM-derived CD34+ cells to differentiate into DC (44). Because IL-10 has potent immunomodulatory properties, including induction of anergy and tolerance in T cells and induction of B cell proliferation and differentiation (12, 45, 46), the fact that mDC2 produced significantly increased levels of IL-10 as compared to mDC1 further indicates that mDC2 are functionally distinct from mDC1.

In summary, we describe a phenotypically and functionally novel monocyte-derived DC subset, mDC2, that skews Th responses towards a Th0/Th2 phenotype. Due to the superior transfection efficiency of mDC2 as compared to mDC1, usage of these cells is an attractive approach to genetic vaccinations and therapies following ex vivo transfections. Because of the unique characteristics of mDC2, lack of IL-12 production and increased IL-10 synthesis in particular, the functional properties of mDC2 in vivo require further studies. Nevertheless, the present data indicate that monocytes have the potential to differentiate into subsets of DC with different cytokine production profiles, which is associated with altered capacity to direct Th cell differentiation.

Furthermore, the mDC2 of the present invention have improved transfection efficiencies compared to the transfection efficiencies of conventional mDC1 cells, as described in greater detail below in “Dendritic Cell Vaccines and Methods of Immunization” and in the Examples.

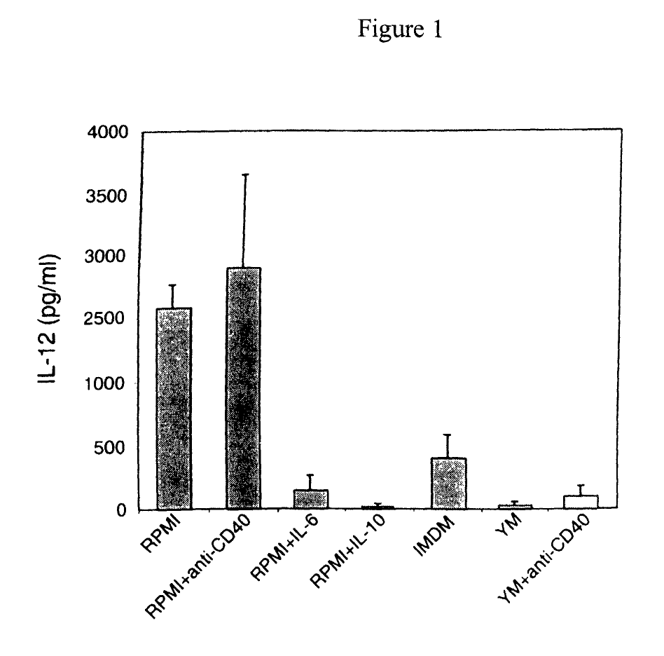

The invention also provides novel dendritic cells exhibiting an intermediate phenotype of CD14⁻ DC with reduced, but detectable, IL-12 production (see FIG. 1, discussed in detail below). Such DC can be generated in the presence of IL-4 and GM-CSF in IMDM (without additional supplements).

Also included are compositions comprising APC and CD1a⁻ dendritic cells of the invention. The CD1a⁻ dendritic cells are capable of presenting an antigen to a T cell. Additionally, in such composition CD1a⁻ dendritic cells may produce substantially no IL-12 and/or promote differentiation of T cells to a Th0/Th2 subtype. In some such compositions, the CD1a⁻ dendritic cells display or present at least one antigen or antigenic fragment thereof. In some such compositions, the at least one antigen or antigenic fragment comprises a protein or peptide differentially expressed on a cell selected from the group consisting of a tumor cell, a bacterially-infected cell, a parasitically-infected cell, and a virally-infected cell, a target cell of an autoimmune response. Such compositions may further comprising a pharmaceutically acceptable carrier, which would be well-known to those of ordinary skill in the art. Certain such compositions may be formulated as a vaccine.

As explained in greater detail below, the mDC2 of the present invention are useful in a wide variety of applications, including antigen-presenting cell therapies or DC therapies. For example, mDC2 are useful in prophylactic and therapeutic dendritic cell therapies, including in vitro, in vivo, and ex vivo applications. In particular, mDC2 are useful in such therapies because the transfection efficiency of these cells is significantly higher than that of conventional mDC1.

APC and DC of the invention (e.g., mDC2) are also useful in applications involving modulation of an immune response, particularly in subjects suffering from autoimmune diseases or disorders. For example, mDC2 are useful in methods for modulating an immune response in a subject having an autoimmune disease or disorder, particularly because mDC2, unlike mDC1, favor Th2 cell differentiation. In one aspect, such methods comprise administering to such subject having a compromised immune system an amount of the mDC2 sufficient to modulate an immune response in the subject. MDC2 of the invention are also useful in applications requiring the display or presenting antigenic proteins or peptides or fragments thereof. For example, given the improved transfection efficiency of mDC2 compared with mDC1, mDC2 are of use in methods for inducing an immune response in a subject by administering to the subject (e.g., following by ex vivo or in vivo transfection of the mDC2 with a nucleic acid encoding an antigenic protein, peptide, or immunogenic fragment thereof or loading of the antigenic protein, peptide, or immunogenic fragment thereof directly into the mDC2, wherein the immune response is desired against the antigenic protein, peptide, or immunogenic fragment thereof) an amount of the mDC2, which displays or presents an antigen or fragment thereof of interest on or at its surface, sufficient to induce an immune response in the subject.

Isolation and Activation of T Cells

T cells are isolated in some embodiments of the invention and activated in vitro (or ex vivo) by contacting the T cell with a dendritic cell of the invention. Several techniques for T cell isolation are known. The expression of surface markers facilitates identification and purification of T cells. Methods of identification and isolation of T cells include flow cytometry, incubation in flasks with fixed antibodies which bind a particular cell type and attachment to magnetic beads.

In one method, density gradient centrifugation is used to separate peripheral blood mononuclear cells, including T cells, from red blood cells and neutrophils according to established procedures. Cells are then washed in an appropriate medium, e.g., PBS, RPMI, AIM-V (GIBCO), and enrichment for T cells is performed by negative or positive selection with appropriate monoclonal antibodies coupled to columns or magnetic beads according to standard techniques. For example, T cells can be isolated by negative selection by depleting CD19, CD14, CD16, and CD56 expressing cells form PBMC using magnetic beads. Following isolation, an aliquot of cells is analyzed for cell surface phenotype including CD4, CD8, CD3, and CD14.

The recovered T cells are then washed and resuspended, and optionally a T cell specific monoclonal antibody, e.g., OKT3, is added to stimulate proliferation.

The proliferative response of T cells in response to an antigen, e.g., presented by the DC of the invention, is generally measured using a mixed lymphocyte response (MLR) assay, antigen-specific T cell lines or clones or peripheral blood T cells specific for the antigen. MLR assays are the standard in vitro assay of antigen presenting function in cellular immunity. The w assay measures the proliferation of T cells after stimulation by a selected cell type. The number of T cells produced is typically characterized by measuring T cell proliferation based on incorporation of ³H-thymidine in culture. Similar methods are used in vivo in nude or SCID mouse models. See also, e.g., Paul (supra); Takamizawa et al. (1997) J Immunology 2134; Uren and Boyle (1989) Transplant Proc 21:208, and 21:3753; Zhou and Tedder (1996) Proc Natl Acad Sci USA 93:2588.

Typically, suspensions of T cells are cultured with allogeneic stimulator cells or autologous DC presenting specific antigens. The stimulator cells, i.e., an antigen presenting cell, such as the DC of the invention, are generally irradiated to prevent uptake of ³H-thymidine. Stimulators and responders are mixed in selected ratios (e.g., 1:1, 1;10, 1;25, &1:50) and plated in e.g., 96 well plates. The cells are cultured together for 5 days, pulsed with thymidine for 18 hours, and harvested. Proliferation of the responder cells is then assessed as a function of thymidine incorporation.

Alternatively, T cell response can be evaluated in a cytotoxic lymphocyte or CTL response. A CTL response is a cell-mediated immune response in which a cytotoxic lymphocyte causes death of a target cell. CTL responses are typically measured by monitoring lysis of target cells by CTLs. An immunogenic peptide or antigenic peptide is a peptide which forms all or a part of an epitope recognized by a T cell (e.g., an epitope which is recognized optionally further includes an MHC moiety), and which is capable of inducing a cell mediated response (including a T helper response). Proteins are processed in antigen presenting cells into antigenic peptides and expressed, e.g., on MHC molecules (or in the context of other molecules such as cell surface proteins) on the surface of antigen presenting cells. Thus, some antigenic peptides are capable of binding to an appropriate MHC molecule on a target cell and inducing a cytotoxic T cell response, e.g., cell lysis or specific cytokine release against the target cell which binds the antigen, or a T helper response. Immunogenic compositions optionally include adjuvants, buffers, and the like.

For example, T cells can be removed from an immunized animal (or human) and tested for their ability to lyse target cells in a CTL assay. Frequently, the target cells are engineered to express one or more of the epitopes contained in the immunogen (e.g., a viral antigen, or a tumor antigen, as described above). The target and effector cells are from the same immunohistocompatibility group (i.e., they have the same MHC components on their surfaces). The target cells are preloaded with a label, typically ⁵¹Chromium, and the T cells, (the effector cells) are then incubated with the target cells for approximately 4 hours. The cultures are then assayed for lysis of the target cells by measuring release of ⁵¹Cr. Alternatively, release of cytoplasmic proteins such as lactose dehydrogenase can be measured, for example using a kit (no. 1644793) made by Boehringer Mannheim (Indianapolis, Indiana). An example of a target cell is a cell transduced with a viral vector encoding a target protein, e.g., a recombinant vaccinia virus vector encoding Gag or Env to test effector cell activity for effectors from animals immunized with a Gag-Env pseudovirus. CTL assays are well-known in the art and protocols can be found in, e.g., Coligan, supra.

In one embodiment, the invention provides a method of inducing or promoting differentiation of T cells, which comprises: co-culturing a population of T cells with a population of APC or dendritic cells of the invention (e.g., mDC2), thereby inducing or promoting T cell differentiation. In one embodiment, the population of APC or dendritic cells comprises a population of greater than about 50%, greater than about 60%, preferably greater than about 70%, preferably greater than about 80%, more preferably greater than about 90%, preferably greater than about 95% CD1a⁻ dendritic cells as described herein. Such populations of CD1a⁻dendritic cells are produced by the methods of the invention.

In some such methods, the T cells comprise naïe T cells. Further, in some such methods, the antigen presenting cell is a CD1a⁻dendritic cell, which may produces substantially no IL-12, or an mDC2. The invention also includes differentiated T cell produced by such methods. In some such methods, the dendritic cell produces substantially no IL-12 compared to a dendritic cell produced by culturing a population of peripheral blood or bone marrow mononuclear cells in IL-4, GM-CSF, and a culture medium comprising RPMI.

Therapeutic and Prophylactic Methods and Applications

Inducing Immune Responses

Methods for modulating an immune response using the dendritic cells of the invention are also a feature of the invention. The dendritic cells of the invention, like conventional dendritic cells are potent antigen presenting cells capable of activating T cells in vitro and in vivo. This feature of the DC of the present invention can be favorably utilized to induce and/or alter a cellular (or organismal) response to an antigen of interest in vitro or in vivo. For example, the DC of the invention are useful activating T cells that recognize an antigen of interest, such as any of the antigens cited herein, including protein or peptide antigens differentially expressed on tumor cells, bacterially-infected cells, parasitically-infected cells, virally-infected cells, as well as antigens expressed by cells that are the target of an autoimmune response and antigens which are the target of an allergic or hypersensitive response. Furthermore, the DC of the invention can be used to induce a prophylactic immune response, in effect, serving as a vaccine for antigens that activate a T cell response, or T-dependent antibody response.

In one aspect, methods for activating T cells ex vivo and in vivo are provided. In some embodiments, dendritic cells or DC progenitors are transfected in vitro with an antigenic peptide or protein. Typically, the sequence encoding the antigenic peptide or protein (subportion of the protein) is operably linked to regulatory sequences, e.g., a constitutive or inducible promoter, enhancers, that are capable of inducing transcription and translation of the peptide, protein, or protein fragment of interest. Alternatively, mature DC produced according to the above described culture procedures are loaded with antigenic peptide without transfection. For example, mDC2 cells can be incubated with synthesized peptide in tissue culture, as described herein. These mDC2 that are transfected with or otherwise loaded with antigenic peptide(s) are then used to activate T cells in vitro, e.g., by co-culturing the DC with naive T cells recovered from the same or a different but compatible subject. Alternatively, the dendritic cells of the invention are introduced into a human or non-human animal subject or recipient to activate T cells in vivo.

The invention also provides an ex vivo method of inducing in a subject a therapeutic or prophylactic immune response against at least one antigen, the method comprising: a) culturing a population of monocytes obtained from the subject with IL-4, GMC-SF, and a culture medium comprising Iscove's Modified Dulbecco's Medium (IMDM) supplemented with insulin, transferrin, linoleic acid, oleic acid and palmitic acid for a sufficient time to produce a population of dendritic cells comprising CD1a⁻dendritic cells; b) introducing to the population of CD1a⁻ dendritic cells a sufficient amount of at least one antigen, or a sufficient amount of an exogenous DNA sequence operably linked to a promoter that controls expression of said DNA sequence, said DNA sequence encoding at least one or said at least one antigen, such that the presentation of the antigen on the CD1a⁻ dendritic cells results; and c) administering the antigen-presenting CD1a⁻ dendritic cells to the subject in an amount sufficient to induce a therapeutic or prophylactic immune response against said at least one antigen. In a preferred embodiment, the culture medium comprises Yssel's medium. The CD1a⁻ dendritic cells are typically mDC2, and are thus distinguished from conventional DC by additional properties and characteristics. Therapeutic or prophylactic amounts can be readily and may comprise amounts equivalent or similar to those utilized in therapeutic or prophylactic treatment methods using conventional DC regimens (e.g., against cancers; see Nestle et al. supra).

A method of therapeutically or prophylactically treating a disease in a subject suffering from said disease is also provided. Such method comprises: a) culturing a population of monocytes obtained from the subject with IL-4, GM-CSF, and a culture medium comprising Iscove's Modified Dulbecco's Medium (IMDM) supplemented with insulin, transferrin, linoleic acid, oleic acid and paimitic acid for a sufficient time to produce a population of CD1a⁻ dendritic cells; b) introducing to the population of CD1a⁻ dendritic cells a sufficient amount of at least one disease-associated antigen, or a sufficient amount of an exogenous DNA sequence operably linked to a promoter that controls expression of said DNA sequence, said DNA sequence encoding at least one of said at least one disease-associated antigen, such that presentation of the disease-associated antigen on the CD1a⁻ dendritic cells results; and c) administering a therapeutic or prophylactic amount of the CD1a⁻dendritic cells presenting the disease-associated antigen to the subject to treat said disease. Preferably, for such methods, the culture medium comprises Yssel's medium. The CD1a⁻ dendritic cells are typically mDC2, and are thus distinguished from conventional DC by additional properties and characteristics.

In addition, the invention provides a method of therapeutically or prophylactically treating a disease in a subject suffering from the disease. Such method comprises: a) culturing a population of monocytes obtained from the subject with IL-4, GM-CSF, and a culture medium comprising Iscove's Modified Dulbecco's Medium (IMDM) supplemented with insulin, transferrin, linoleic acid, oleic acid and palmitic acid for a sufficient time to produce a population of CD1a⁻ dendritic cells; b) contacting the population of CD1a⁻ dendritic cells with a population of diseased cells from a tissue or organ of the subject, thereby inducing presentation of a disease-associated antigen on the CD1a⁻ dendritic cells; and c) administering a therapeutic or prophylactic amount of CD1a⁻ dendritic cells presenting the disease-associated antigen to the subject to treat the disease. In a preferred embodiment, the culture medium is Yssel's medium, and the CD1a⁻ dendritic cells are mDC2.

A disease-associated antigen is one that is associated with a disease or disease state (e.g., of a cell or organism), or is involved in causing a cell to become diseased. A variety of disease-associated antigens are known, including those antigens associated with diseases described previously.

For such therapeutic and prophylactic treatment methods, therapeutic or prophylactic amounts can be readily determined by one of ordinary skill in the art. For example, such amounts may be equivalent or similar to those utilized in therapeutic or prophylactic methods employing conventional DC regimens (e.g., against cancers; see Nestle et al. supra).

T cells such as CD8⁺ CTLs activated in vitro are introduced into a subject where they are cytotoxic against target cells bearing antigenic peptides that the T cell recognizes on MHC class I molecules. These target cells are typically cancer cells or infected cells which express unique antigenic peptides on their MHC class I surfaces.

Similarly, helper T cells (e.g., CD4⁺ T cells), which recognize antigenic peptides in the context of MHC class II, are also stimulated by the recombinant DC, which comprise antigenic peptides both in the context of class I and class II MHC. These helper T cells also stimulate an immune response against a target cell. As with cytotoxic T cells, helper T cells are stimulated with the recombinant DC in vitro or in vivo.

The dendritic cells and T cells are preferably isolated from the same individual into which the activated T cells are to be active (“autologous” therapy). Alternatively, the cells can be those from a donor or stored in a cell bank (e.g., a blood bank). For therapeutic and prophylactic purposes, the activated T cells, e.g., autologous T cells activated in vitro with mDC2 displaying an antigen of interest produced either by introducing and expressing an exogenous DNA encoding the peptide of interest, or externally loading the peptide of interest, are then administered to the subject in an amount sufficient to produce a measurable immune response. For example, to produce an enhanced response against a tumor, peripheral blood monocytes are isolated from a subject, e.g., a human subject with the tumor, and differentiated in vitro according to the methods described above. The differentiated DC are transfected, or otherwise caused to display (present) an antigen expressed by the tumor. Circulating naive T cells are similarly recovered from the subject and contacted with the DC in vitro, resulting in activation of T cells specific for the tumor antigen. The T cells (or a mixed population including both DC and T cells) are then reintroduced into the subject, where they are capable of effecting a specific immune response against the tumor in vivo.

The dendritic cells of the invention, once transfected or loaded to present an antigen of interest, can also be administered directly to a subject to produce T cells active against a selected, e.g., cancerous or infected, cell type. Administration is by any of the routes normally used for introducing a cell into contact with a subject's blood or tissue cells.

In addition, the DC of the invention can also be used to modulate, rather than activate, a specific immune response. In certain disease conditions, most notably autoimmune responses (e.g., rheumatoid arthritis, lupus erythematosous) and transplant rejection, the balance between Th1 and Th2 effector cells is critical to the expression and progression of the disorder. Because the dendritic cells of the invention promote Th0/Th2 lineage development, and deter Th1 lineage development, activation of naive T cells in vitro or in vivo with mDC2 can be used to modulate the immune response towards a Th2 response, thus ameliorating symptoms and progression of such disease states. For example, the dendritic cells of the invention can be utilized as a transplant prophylaxis. Antigens corresponding to, or derived from the tissue to be transplanted are loaded on mDC2. The mDC2 displaying transplant specific antigens are then administered to the transplant recipient. Alternatively, the mDC2 cells are used to activate autologous T cells in vitro, and the T cells reintroduced into the subject. Typically, such a procedure precedes, or is conducted concomitant, with the tissue transplant.

The cells are administered to a subject in any suitable manner, often with pharmaceutically acceptable carriers. Suitable methods of administering cells in the context of the present invention to a subject (such as a patient) are available, and although more than one route can be used to administer a particular cell composition, a particular route can often provide a more immediate and more effective reaction than another route. For the purposes of the present invention, a subject can be either human (such as a patient or experimental subject) or a non-human animal, such as a mammal, including a primate, a mouse, a hamster, a rat, or other laboratory animal, companion animal (e.g., dog, cat) or domestic livestock (e.g., cow, horse, goat, sheep, etc.) or other vertebrate.

Pharmaceutically acceptable carriers are determined in part by the particular composition being administered, as well as by the particular method used to administer the composition. Accordingly, there is a wide variety of suitable formulations of pharmaceutical compositions of the present invention. Most typically, quality controls (e.g., microbiology, clonogenic assays, viability assays), are performed and the cells are reinfused back to the patient. See Korbling et al. (1986) Blood 67:529; and Hass et al. (1990) Exp Hematol 18:94.

Formulations suitable for parenteral administration, such as, for example, by intraarticular (in the joints), intravenous, intramuscular, intradermal, intraperitoneal, intratumor, and subcutaneous routes, and carriers include aqueous isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Intravenous, subcutaneous and intraperitoneal administration are the preferred method of administration for dendritic or T cells of the invention.

The dose of cells (e.g., activated T cells, or dendritic cells) administered to a patient, in the context of the present invention should be sufficient to effect a beneficial therapeutic response in the patient over time, or to inhibit growth of cancer cells, or to inhibit infection. Thus, cells are administered to a patient in an amount sufficient to elicit an effective cell mediated response to a virus or tumor, or infected cell, and/or to alleviate, reduce, cure or at least partially arrest symptoms and/or complications form the particular disease or infection. An amount adequate to accomplish this is defined as “therapeutically effective dose.” The dose will be determined by the activity of the T cell or dendritic cell produced and the condition of the patient, as well as the body weight or surface area of the patient to be treated. The size of the dose also will be determined by the existence, nature, and extent of any adverse side-effects that accompany the administration of a particular cell in a particular patient. In determining the effective amount of the cell to be administered in the treatment or prophylaxis of diseases such as AIDS or cancer (e.g., metastatic melanoma, prostate cancer, etc.), the physician needs to evaluate circulating plasma levels, cytotoxic lymphocyte or helper toxicity, progression of the disease, and the production of immune response against any introduced cell type.

Prior to infusion, blood samples are obtained and saved for analysis. Generally at least about 10⁴ to 10⁶ and typically, between 1×10⁶ and 1×10⁸ cells are infused intravenously or intraperitoneally into a 70 kg patient over roughly 10-120 minutes. Intravenous infusion is preferred. Vital signs and oxygen saturation are closely monitored. Blood samples are obtained at intervals and saved for analysis. Cell reinfusion can be repeated approximately weekly or monthly, over a period of up to approximately 1 year. Such procedures can be performed on an inpatient or outpatient basis at the discretion of the clinician.

For administration, cells of the present invention (DC or activated T cells) can be administered at a rate determined by the LD-50 (or other measure of toxicity) of the cell type, and the side-effects of the cell type at various concentrations, as applied to the mass and overall health of the patient. Administration can be accomplished via single or divided doses. The cells of this invention can supplement other treatments for a condition by known conventional therapy, including cytotoxic agents, nucleotide analogues and biologic response modifiers. Similarly, biological response modifiers are optionally added for treatment by the DC or activated T cells of the invention. For example, the cells are optionally administered with an adjuvant, or cytokine such as GM-CSF, or IL-2. Doses will often be in the range of 1×10⁵ to 1×10⁷ cells per administration.

Regardless of whether the DC of the invention are used in vitro or in vivo to stimulate T cell responses, the relevant antigen can be loaded externally, or expressed following introduction, e.g., transfection, into the DC as described above.

Dendritic Cell Vaccines and Immunization Methodologies

Genetic vaccinations are a very promising new approach for vaccine research and development. Direct transfection of DC in vivo has been shown to be essential for the induction of immune response after genetic vaccinations (Akbari et al. (1999) J. Exp. Med. 189:169). In addition, ex vivo transfection of DC is a promising approach in therapeutic applications (Liu (1998) Nat. Biotechnol. 16:335), and DC loaded with the relevant antigen have been shown to induce protective immune responses in several animal models of infectious and malignant diseases (Ashley et al. (1997) J. Exp. Med. 186:1177; Ludewig et al. (1998) J. Virol. 72:3812). DC pulsed or transfected ex vivo with the desired antigens are currently undergoing investigation in clinical trials as a means to induce pathogen or tumor specific immune responses (Nestle et al. (1998) Nat. Med. 4:269; Kundu et al. (1998) AIDS Res. Hum. Retroviruses 14:551). Until now, the low transfection efficiencies of DCs have reduced the efficacy of gene transfer approaches using plasmid DNA. However, plasmid DNA vectors provide several advantages over alternate vector technologies, such as excellent stability and ease of manufacturing and quality control (Liu (1998) Nat. Biotechnol. 16:335). mDC2 are a promising target for DC therapies, because the transfection efficiency of these cells is significantly higher than that of mDC1. The transfection efficiency of mDC2, which in this study was an average 3.5%, exceeds that of conventional DC transfected with the gene gun (Timares et al. (1998) Proc. Natl. Acad. Sci. USA 95:13147). Transfection efficiencies of only 0.1% to 2.2% were obtained in murine dendritic cell lines transfected with the gene gun (Timares et al., supra), although the technology typically allows efficient transfection efficiencies due to direct delivery of DNA into the nucleus of the cells. The transfection efficiency obtained by viral vectors is typically significantly higher than those obtained by naked DNA vectors (Arthur et al. (1997) Cancer Gene Therapy 4:17; Szabolcs et al. (1997) Blood 90:2160; Zhong et al. (1999) Eur. J. Immunol. 29:964). However, the viral proteins expressed by adenovirus-infected DC also activate virus-specific CTLs resulting in lysis of the transfected DC (Smith et al. (1996) J. Virol. 70:6733), which is likely to reduce the efficacy of viral vectors in therapeutic applications. Because of the potent antigen-presenting cell function of DC, significant immune responses have been generated in vivo following transfer of DC transfected using either chemical methods or by gene gun, despite the low transfection efficiencies of the cells (Alijagic et al. (1995) Eur. J. Immunol. 25:3100; Manickan et al. (1997) J. Leukocyte Biol. 61:125; Timares, supra). Because of their superior transfection efficiency, we are currently using mDC2 to screen libraries of genetic vaccine vectors and immunomodulatory molecules generated by recursive sequence recombination methods, e.g., DNA shuffling (see, e.g., Crameri et al. (1998) Nature 391:288; Chang et al. (1999) Nat. Biotechnol. 17:793), to identify variants that are optimized for DC. In addition, improved transfection efficiency of mDC2 as compared to conventional mDC1 makes them an attractive means to generate DC-based vaccines, particularly in applications when Th0/Th2 responses are desired.

Dendritic cell vaccines utilizing the monocyte-derived APC or mDC2 of the present invention are useful for cancer immunotherapies, including in therapeutic and prophylactic treatment regimens for the following cancers: prostate cancer; non-Hodgkin's lymphoma; colon cancer; breast cancer; leukemia; melanoma; brain, lung, colorectal, and pancreatic cancers; renal cell carcinoma; and lung, colorectal, pancreatic B-cell lymphoma, multiple myeloma, prostate carcinomas, sarcomas, and neuroblatomas, including those cancers described in Timmerman et al. (1999) Annu. Rev. Med. 50:507-29. The antigens for such cancers are present in Timmerman et al., id. at 523. Such antigens can be presented or displayed on the APC or mDC2 of the invention (using peptide loading, pulsing or transfection methods described above).

The invention provides vaccines and compositions comprising an mDC2 (derived from the monocytes) that displays or presents an antigen to the cancer (or other disease or disorder) to be treated. A dendritic cell vaccine of the invention typically comprises an mDC2 that displays or presents an antigen to the cancer (or other disorder) in combination with a carrier, (e.g., pharmaceutically acceptable carrier) and other additives, if desired, that facilitate the vaccination treatment method or strategy.

Vaccination regimens and immunotherapeutic strategies against cancers are typically performed using ex vivo methods. In brief, in one aspect, the invention provides methods comprising removing or isolating a population of monocytes from a subject (e.g., animal or human) to be treated for a particular cancer, growing the monocytes in vitro and using the methods of the invention as described above to generate mDC2 from the monoctyes, and exposing or contacting the mDC2 (or differentiating monoctyes) with a population of cancer cells from the subject for a sufficient time and under sufficient conditions, as described above with regard to antigen presentation, such that the mDC2 display or present an antigen to the cancer. The antigen-presenting mDC2 are typically washed thoroughly 3× in, e.g., sterile PBS, to remove media and other components. They are then re-suspending in PBS or other appropriate carrier and then immediately administered or delivery to the subject in appropriate, using standard methods for administration or delivery of dendritic cells to a tissue or organ site of interest (e.g., the site of cancer) as are used with conventional dendritic cells in conventional dendritic cell therapies. See, e.g., Nestle et al. (1998) Nature Medicine 4:328, which is incorporated herein by reference in its entirety for all purposes.

Vaccination regimens and strategies using mDC2 vaccines, including dosages, are analogous to known regimens and strategies using conventional dendritic cell vaccines. The specific methodology to be employed with mDC2 vaccines can be modeled after ex vivo dendritic cell vaccination approaches currently utilized with conventional mDC1 and known to those of ordinary skill in the art. For example, vaccine regimens for cancers (e.g., melanoma), with booster immunizations, using an mDC2 vaccine or composition of the invention comprising an mDC2 that presents at least one appropriate antigen, can be performed as described in Nestle et al. (1998) Nature Medicine 4:328. For example, direct delivery of antigen-displaying or antigen-presenting mDC2 (in which the antigen of interest has been delivered to the mDC2 via peptide loading or transfection with a nucleic acid encoding the antigen of interest) (1×10⁶ cells per injection) to a subject can be performed, e.g., by delivery of an initial dose followed by daily or weekly injections (e.g., into a professional lymphoid organ, a peripheral tissue site (e.g., skin) or intravenously) for one or more months. Booster immunizations can be repeated following this initial immunization period after two weeks and thereafter, if desired, in monthly intervals. See id.

As discussed above, the mDC2 of the invention are also useful in vaccination and immunotherapeutic regimens and approaches against other diseases and disorders, including, e.g., viral diseases and disorders, e.g., hepatitis B and C virus, herpes simplex virus, Epstein-Barr virus, human immunodeficiency virus (HIV), human papilloma virus (KPV), Japanese encephalitis virus, dengue virus, hanta virus, Western encephalitis virus, polio, measles, and the like; and diseases and disorders relating to bacterial (e.g., pneumonia, staph infections) and mycobacterial (e.g., for TB, leprosy, or the like); allergies (e.g., relating to house dust mite, storage dust mite, grass allergens); Malaria from Plasmodium sp. (including P. falciparum, P. malariae, P. ovale, and P. vivax; including viral, bacterial, allergic, autoimmune (such as, e.g., multiple sclerosis, Rheumatoid arthritis, juvenile diabetes mellitus, psoriasis, certain arthridities, and the like) parasitic, inflammatory, infectious, hyperproliferative, contraception, and cancer diseases and disorders listed in PCT Application Publication No. WO 99/41383, published Aug. 19, 1999. For these diseases and disorders, the vaccination regimens, methods, and strategies are analogous or similar to those currently employed with conventional dendritic cells. One of ordinary skill in the art can readily design a specific vaccination method and strategy for a particular disease or disorder based upon strategies used with conventional mDC1.

The present invention also provides an ex vivo method of modulating or inducing an immune response in an immunocompromised subject, including a subject suffering from an autoimmune or inflammatory disease or disorder, or the like. The mDC2 of the invention are useful in modulating an immune response in such an immunocompromised subject. In one aspect, the invention provides a method comprising removing or isolating a population of monocytes from an immunocompromised subject, growing the monocytes in vitro using the methods of the invention described herein such that mDC2 are generated, and then administering or delivering the resulting mDC2 to the subject in an amount sufficient to modulate or induce an immune response. Methods for administration or delivery, including dosages and immunization regimens and strategies (including booster immunizations) similar or equivalent to those described above for cancer immunotherapy can be employed.

Use of Dendritic Cells as Adjuvants

The antigen presenting cells and mDC2 of the present invention are also useful as adjuvants. They act as adjuvants in enhancing the immune response to an antigen. In particular, they prime T cells in the absence of any other adjuvant. Like conventional DC, the antigen presenting cells and mDC2 of the invention act as adjuvants based on the following functional characteristics: potency (e.g., small numbers of mDC2 pulsed with lose doses of antigen stimulate strong T-cell response); primary response (e.g., naïve and quiscent T cells can be activated with antigens on mDC2); and physiology (CD4⁺ T helpers and CD8⁺ T killers are primed in vivo and ex vivo). See Paul, supra, pp. 550-551. For a more complete description of DCs as adjuvants, see id.

The invention provides methods for enhancing or modulation an immune response comprising administration to a subject of an amount of an mDC2 sufficient to enhance or modulate an immune response to at least one antigen. The mDC2 are produced from monocytes isolated or removed from the subject to be treated, as described above with regard to cancer immunotherapies and therapies with immunocompromised subjects (e.g., subjects having autoimmune disorders). A population of mDC2 is administered or delivered to the subject (depending on the application, with or without at least one antigen of interest presented on or at the mDC2 surface), as described above, in an amount sufficient to enhance immunity or modulate an immune response to the at least one antigen. Standard adjuvants may also be used in such methods to enhance immunity. In this way, it may be possible to increasing the access of antigens to mDC2 or the function of mDC2. Paul, supra, p. 551.

Assays and Kits

The present invention provides commercially valuable in vitro, ex vivo, and in vivo assays and kits to practice the assays. In the assays of the invention, mDC2 are transfected or otherwise caused to present a putative T cell antigen. The mDC2 is used to activate the T cell, which is then assayed for a proliferative or cytotoxic response (e.g., in a MLR or CTL assay). Because the transfected mDC2 cells can be established in culture, in vitro or ex vivo, or made in batches, several potential target cell populations can be screened. Thus libraries of potential e.g., tumor antigens can be screened by cloning into the dendritic cells of the invention. The ability to screen and identify tumor and pathogen derived antigens is of considerable commercial value to pharmaceutical and other drug discovery companies.

Kits based on such assays are also provided. The kits typically include a container, and monocytes or dendritic cells. The kits optionally comprise directions for performing the assays, cell transfection vectors, cytokines, or instructions for the use of any of these components, or the like.

In a further aspect, the present invention provides for the use of any composition, cell, cell culture, apparatus, apparatus component or kit herein, for the practice of any method or assay herein, and/or for the use of any apparatus or kit to practice any assay or method herein and/or for the use of cells, cell cultures, compositions or other features herein as a therapeutic formulation. The manufacture of all components herein as therapeutic formulations for the treatments described herein is also provided.

### EXAMPLES

The following examples are provided by way of illustration only and not by way of limitation. Those of skill will readily recognize a variety of noncritical parameters which can be changed or modified to yield essentially similar results. Reagents suitable for the practice of the present invention are commercially available from a variety of sources, and will be readily apparent to those of skill in the art.

In these examples, the reagents and cell cultures were obtained from the following sources: Purified recombinant human IL-4, IL-10, IFN-γ, M-CSF, and TNF-α were obtained from R&D Systems (Minneapolis, MN), and GM-CSF was obtained from Schering-Plough, Inc. (County Cork, Ireland). Fluoescein-5′-isothiocyanate-(FITC-) or phycoerthyrin-(PE-) conjugated monoclonal antibodies (mAbs) specific for CD1a, CD3, CD11b, CD11c, CD13, CD14, CD16, CD19, CD23, CD28, CD33, CD40, CD54, CD56, CD64, CD80, CD86, HLA-DR and HLA-ABC were purchased from PharMingen (San Diego, Calif.), and PE-conjugated anti-CD83 mAb was obtained from Coulter (Miami, FL). RPMI-1640 and Iscove's modified Dulbecco's medium (IMDM) were obtained from Life Technologies (Rockville, Md.) (Gibco BRL Life Technologies Products & Reference Guide 2000-2001 Catalog No. 21056; 1×liquid mg/L; p. 1-52).

Yssel's medium was IMDM enriched with insulin (5 μg/ml, Sigma, St. Louis, Mo.); human transferrin (20 μg/ml, Boehringer Mannheim, Mannheim, Germany); linoleic acid (2 μg/ml, Sigma); oleic acid (2 μg/ml, Sigma); palmitic acid (2 μg/ml, Sigma); BSA (0.25% (w/v), Sigma); 2-amino ethanol (1.8 mg/L, Sigma), as described in Yssel et al. (1984) J Immunol Methods 72(1):219.

All media were also supplemented with 10% fetal bovine serum (Hyclone, Logan, UT), 2 mM glutamine, 50 U/ml penicillin, and 100 μg/ml streptomycin.

Histopaque was from Sigma Corp., and immunomagnetic beads coated with anti-mouse antibodies (Abs) (Dynabeads P-450) were purchased from Dynal (Oslo, Norway).

### Example 1

### Differentiation of Novel Subtypes of Dendritic Cells in Culture

Dendritic cells with novel cytokine production profiles, improved transfection properties, and altered capacity to direct Th cell differentiation were generated after culture in vitro by the methods of the invention. Materials and methods for the generation of the novel antigen-presenting cell subtypes are described in detail below. Such materials and methods can also be employed to generate such APC subtypes ex vivo or in vivo in the cells, tissues, and/or organs of subjects.

1. Cell Preparations and Culture Conditions

Peripheral blood was obtained from healthy blood donors as standard buffy coat preparations collected at Stanford University Medical School Blood Center (Palo Alto, Calif.). Peripheral blood mononuclear cells (PBMC) were isolated by a Histopaque density-gradient centrifugation and washed twice with PBS (phosphate-buffered saline) at +4° C. Monocytes were purified by negatively depleting T, B and NK cells using mouse-Ab reactive immunomagnetic beads (Dynal, Oslo, Norway). Anti-CD3-, anti-CD16-, anti-CD19- and anti-CD56-labeled PBMCs were incubated with the beads for 30 min at 4° C. with gentle rotation, and positive cell were removed by a Dynal magnet. After washing in PBS containing 2% FBS, purified monocytes were collected and counted. Allogeneic T cells were isolated by negative selection by depleting CD19-, CD14-, CD16-, and CD56-expressing cells from PBMC using magnetic beads. Purified T cells were cryopreserved and thawed to be used in coculture experiments. To generate DC, purified monocytes (1×10⁶/ml) were cultured in 12-well culture plates (Costar, Cambridge, Mass.) in a final volume of 1.5 ml. Recombinant human IL-4 (400 U/ml) and GM-CSF (800 U/ml) were added to the cultures, and half of the medium was replaced after every two days with fresh media containing IL-4 and GM-CSF at final concentrations of approximately 400 U/ml and 800 U/ml, respectively. All cell cultures were performed at 37° C. in humidified atmosphere containing 5% CO₂ in RPMI (Life Technologies, Rockville, Md.), IMDM, or Yssel's medium supplemented with 10% FBS, 2 mM glutamine, 50 U/ml penicillin and 100 μg/ml streptomycin. When indicated in the text, anti-human CD40 mAb (10 μg/ml) or TNF-α (100 nanogram/milliliter (ng/ml)) was added on day 5, and/or LPS (1 ng/ml; Sigma) plus IFN-γ (10 ng/ml) were added on day 6. After 7 days of culture, DC were harvested and used in the experiments.

2. Flow Cytometry

Flow cytometry can be used according to protocols well known in the art (see, e.g., Coligan et al. (eds.)(1991) Current Protocols in Immunology, Wiley and Sons, Inc. (New York)), to characterize the dendritic cells produced according to the methods of the present invention. Specifically, cells were washed twice with PBS supplemented with 2% FCS containing 0.01% sodium azide. FITC- and PE-conjugated mAbs were added at saturating concentrations for 30 min at 4° C., and two additional washes were performed. FITC- or PE-conjugated mAbs specific for CD1a, CD14, CD40, CD80, CD86, HLA-DR, HLA-A,B,C, CD11b, CD11c, CD13, CD33, CD23, CD54, CD64, and CD83 were used to label the cells. Goat anti-mouse Abs (FITC- or PE-conjugated) with no known reactivity to human antigens were used as negative controls. Cell surface antigen expression was evaluated by single or double immunofluorescence staining and analysis was performed using a FACScalibur flow cytometer and CellQuest software (Becton Dickinson, San Jose, Calif.).

3. Analysis of Cytokine Levels in Culture Supernatants

Supernatants of DC and T cell cultures were stored at −80° C. until they were analyzed for the presence of cytokines. The cytokine production profiles of mature mDC1 and mDC2 were essentially the same as those of the corresponding CD83⁻ subsets, demonstrating that the cytokine production profiles of mDC1 and mDC2 remain stable upon maturation. Cytokine levels in mature mDC1 and mDC2 supernatants were determined using cytokine-specific ELISAs. IL-2, L-4, IL5, IL-6, IL-8 IL-10, IL-13, and IFN-γ levels were determined using commercially available kits (R&D Systems). IL-12 levels were measured using ELISA based on paired IL-12-specific Abs (MAB611, BAF219), and the assays were performed according to the manufacturer's instructions (R&D Systems).

4. T Cell Differentiation Assays

Autologous T cells (1×10⁶ cells/well) were co-cultured with either mDC1 or mDC2 (1×10⁵ cell/well) generated as described above in 24-well culture plates (Costar) for 5 days in Yssel's medium. T cells were harvested and stimulated with 1 μg/ml of anti-CD3 mAb and 10 μg/ml of anti-CD28 mAb for 24 hours. The supernatants were then harvested and the concentrations of cytokines were measured by cytokine-specific ELISAs, as described above, using commercially available kits (R & D Systems).

5. Statistical Analysis

Statistical analysis was performed using the Student's t test (two-tailed) in this Example and the Examples presented below. Values of p<0.05 were considered significant in all Examples.

6. Results

DC were differentiated from PB monocytes in the presence of IL-4 and GM-CSF, as described by Sallusto et al. (1994) J. Exp. Med. 179:1109, and a variety of cytokines and growth factors was studied to identify conditions that favor the differentiation of DC with altered cytokine production profiles.

When RPMI was used as the culture medium, supplemented with IL-4 and GM-CSF, conventional DC producing high levels of EL-12 were generated, which is consistent with previous studies (Macatonia et al. (1995) J. Immunol. 154:5071; Koch et al. (1996) J. Exp. Med. 18:741; and Rissoan et al. (1999) Science 283:1183). Both IL-6 and L-10 inhibited EL-12 production by DC. However, the cells cultured in the presence of IL-6 or IL-10 remained CD 14⁺, indicating that these cytokines also prevented DC differentiation (data not shown).

In contrast, when PB monocytes were cultured in the presence of Yssel's medium (IMDM supplemented with insulin, transferrin, linoleic acid, oleic acid, and palmitic acid) supplemented with IL-4 and GM-CSF as described above, for approximately seven days, monocytes differentiated into CD14⁻ dendritic cells, which exhibited an altered cytokine production profile. In particular, such CD14⁻ dendritic cells virtually completely lacked EL-12 production upon activation by LPS and IFN-γ. See FIG. 1, which illustrates IL-12 production by DC generated under different culture conditions. IL-12 production was absent or minimal also when cultured in the presence of cross-linked anti-CD40 mAbs (10 μg/ml) and subsequently activated with LPS and IFN-γ (FIG. 1).

Relative IL-12 production by DC generated under the culture conditions described above is shown in FIG. 1. PB monocytes were cultured in the presence of IL-4 (400 U/ml) and GM-CSF (800 U/ml) in either RPMI (n=15), IMDM (n=4) or Yssel's medium (n=14). In some cultures, IL-6 (100 U/ml) (n=3) or IL-10 (100 U/ml) (n=4) were added at the onset of the cultures, or anti-CD40 mAbs (10 μg/ml) were included on day 5 (n=11) and studied as indicated in the FIG. 1. After a culture period of six days, the cells were harvested and activated with LPS (1 (ng/ml)) plus IFN-γ (10 ng/ml). The supernatants were harvested after culturing for an additional 24 hours, and the levels of IL-12 in the supernatants were measured by ELISA. The results are expressed as mean±SEM.

If monocytes were cultured in unsupplemented (plain) IMDM in the presence of IL-4 and GM-CSF, an intermediate phenotype of CD14⁻ dendritic cells resulted, characterized by reduced, but detectable, IL-12 production (FIG. 1).

Each of the components of Yssel's medium, namely insulin, transferrin, linoleic acid, oleic acid, and palmitic acid, has been shown to affect the function of lymphoid cells in vitro and/or in vivo (see, e.g., Lernhardt (1990) Biochem. Biophys. Res. Commun. 166:879; Wooten et al. (1993) Cell. Immunol. 152:35; Karsten et al. (1994) J. Cell. Physiol. 161:15; Okamoto et al. (1996) J. Immunol. Meth. 195:7; and Kappel et al. (1998) Scand. J. Immunol. 47:363). To further characterize the culture conditions that favor mDC2 differentiation, we added individual components of Yssel's medium to RPMI, and analyzed IL-12 production and CDla expression. In addition, because IMDM differs from RPMI in that it contains higher concentrations of glucose, and because glucose has been shown to influence cytokine production by monocytes, with higher glucose concentrations enhancing cytokine production (see, e.g., Morohoshi et al., (1996) “Glucose-dependent interleukin 6 and tumor necrosis factor production by human peripheral blood monocytes in vitro,” Diabetes 45:954), we also studied the effect of glucose on differentiation of DC. Addition of glucose at concentrations 4.5 mg/ml and 9.0 mg/ml did not significantly alter or inhibit (n=2) EL-12 production by conventional DC generated in RPMI (compared to DC generated in Yssel's medium), whereas a combination of linoleic acid, oleic acid, and palmitic acid inhibited, but never completely blocked, CDla expression on mDC1 (data not shown). Nevertheless, under the experimental conditions described herein, no single component of Yssel's medium was able to fully substitute the effect of the complete medium in inducing altered cytokine production in differentiated DC cells (i.e., differentiation of mCD2) (data not shown). Moreover, if the monocyte cultures were initiated with RPMI, and Yssel's medium was added after 24 hours after the onset of the cultures, the cells differentiated into conventional MDC1 producing high levels of IL-12 upon activation (data not shown), demonstrating that DC differentiation into subsets with different cytokine production profiles is dependent on a delicate balance of growth factors that are present during the initial stages of DC differentiation.

### Example 2

### Phenotypic Characterization of Dendritic Cells Producing High or Low Levels of IL-12

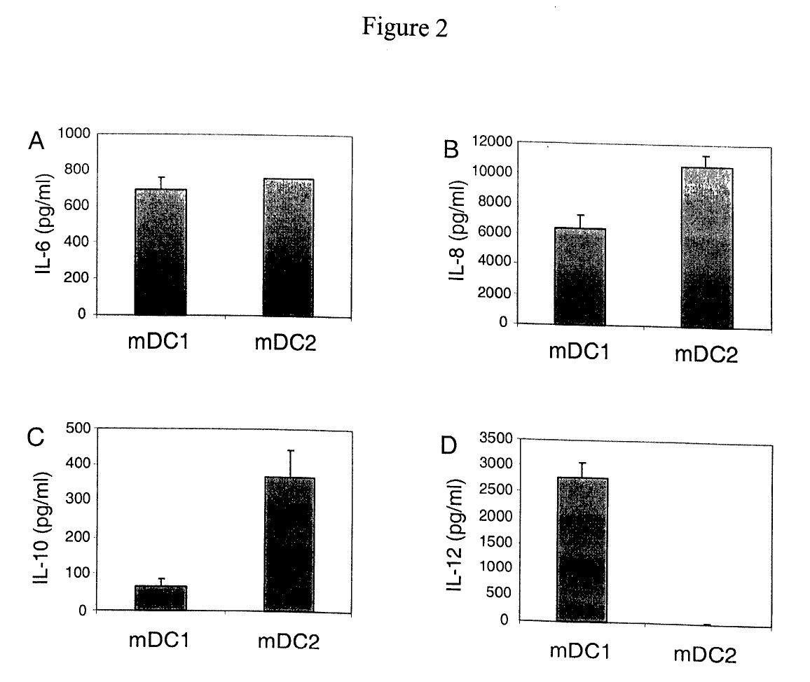

To analyze whether the lack of IL-12 production by DC cultured in the presence of Yssel's medium was associated with altered expression of cell surface antigens, phenotypic characterization of the cells was performed by using flow cytometry as described above in Example 1. Monocytes that were differentiated in Yssel's medium had the typical morphologic appearance of dendritic cells and expressed markers characteristic of DC, such as, e.g., CD11c, CD40, CD80, CD86, and MHC class II, as shown in FIG. 2, which illustrates the phenotypic characterization of DC generated in the presence of RPMI or Yssel's medium. Freshly isolated monocytes (A), or DC differentiated in the presence of IL-4 (400 U/ml) and GM-CSF (800 U/ml) in RPMI (B) or Yssel's medium (C) were harvested and stained with mAbs (as indicated in FIG. 2). The expression levels of the corresponding antigens were analyzed using a FACScalibur flow cytometer.

No significant difference in the mean fluorescence intensity (MFI) of these antigens was observed irrespective of whether the cells were differentiated in the presence of RPMI or Yssel's medium. In addition, no differences in the expression levels of CD13, CD23, CD32, CD33, CD54, and MHC class I molecules between these DC populations were observed, and both subsets (subtypes) also expressed CD47 (data not shown). Furthermore, the DC differentiated either in the presence of Yssel's medium or RPMI strongly downregulated expression of CD14 (as an indication of differentiation into DC) (FIG. 2), demonstrating a phenotype of conventional DC. As a control, monocytes differentiated in the presence of GM-CSF in either medium differentiated into macrophages expressing high levels of CD14 with macroscopic appearance of macrophages (data not shown).

However, in contrast to DC cultured in the presence of RPMI, DC cultured and differentiated in the presence of Yssel's medium consistently expressed minimal or no CDla (FIG. 2). This finding was consistently observed in 12 separate experiments, suggesting that IL12 and CD1a may be regulated by similar mechanisms. To distinguish dendritic cell populations with these differences in IL-12 production and CD1a expression, the conventional CD1a⁺ DC were designated MDC1, whereas CD1a⁻ DC lacking IL-12 production were designated mDC2.

### Example 3

### MDC2 Produce Increased Levels of IL-10 Compared to Conventional MDC1

To further study the cytokine production profile of the novel DC of the present invention (e.g., mDC2), and to exclude the possibility that low or lack of IL-12 production related to a generally poor response or non-specific reduction in response of the cells to activation, the capacity of mDC2 cells to respond to activation by producing IL-6, IL-8 and IL10 was evaluated. mDC1 and mDC2 derived from the same donor were activated with LPS and IFN-γ for 24 hours. Supernatants were collected and cytokine levels were determined by using cytokine-specific ELISA as described above.

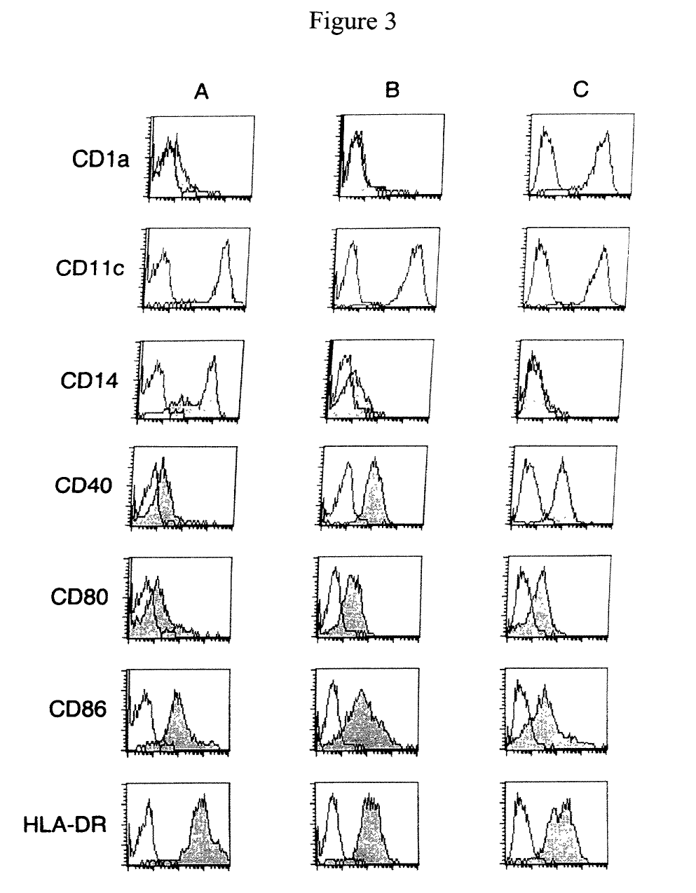

Cytokine production profiles of mDC1 and mDC2 are shown in FIG. 3. DC were generated in the presence of IL-4 (400 U/ml) and GM-CSF (800 U/ml) in either RPMI (mDC1) or Yssel's medium (mDC2). DC were harvested after a culture period of six days, the cells were cultured for an additional 24 hours in the presence of LPS (1 ng/ml) plus IFN-γ (10 ng/ml). The supernatants were harvested and the levels of (A) IL-6 (n=6), (B) IL-8 (n=8), (C) IL-10 (n=5), and (D) IL-12 (n=15) were measured by cytokine-specific ELISA. DC subsets from the same donors were analyzed in parallel, and the results are expressed as mean±SEM.

As shown in FIG. 3, MDC1 and mDC2 derived from the same donors produced comparable levels of IL-6 and IL-8, whereas IL-12 production was consistently absent in cultures of mDC2. MDC2 produced significantly higher levels of IL-10 than mDC1 (FIG. 3), further supporting the conclusion that mDC1 and mDC2 are functionally separate DC subsets (or subtypes). However, it is clear that IL-10 was not the underlying mechanism inducing differentiation of mDC2, because DC cultured in the presence of exogenous IL-10 (100 U/ml) remained CD14⁺, which is consistent with a previous study indicating that IL-10 promotes differentiation of peripheral blood monocytes into macrophages (Allavena et al. (1998) “IL-10 prevents the differentiation of monocytes to dendritic cells but promotes their maturation to macrophages,” Eur J Immunol 28, no. 1:359).

### Example 4

### Maturation of MDC2 into CD83⁺ Cells

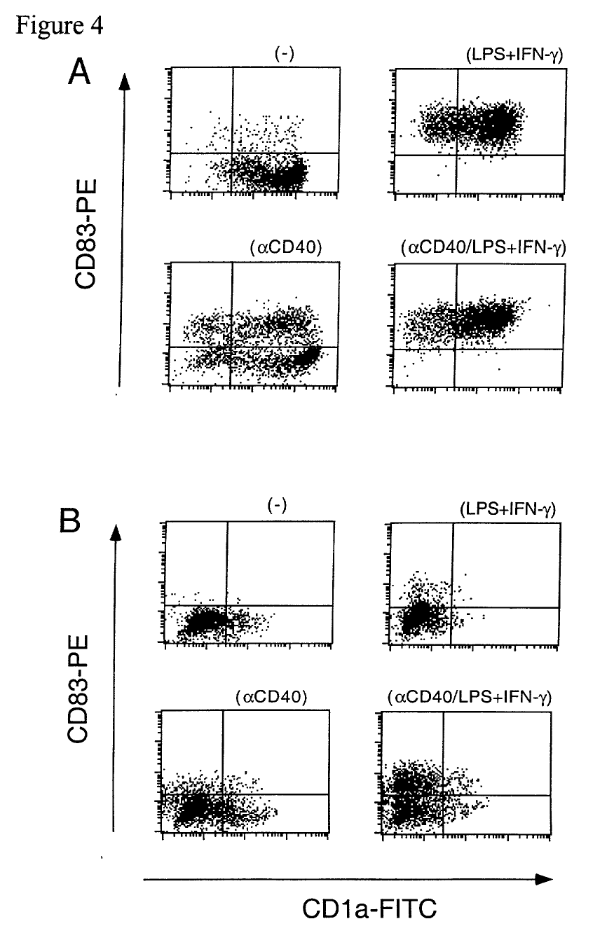

Several activation signals, such as anti-CD40 monoclonal antibodies (mAbs), CD40 ligand (CD154), TNF-α, or a combination of LPS and IFN-γ, can induce maturation of conventional monocyte-derived DC, mDC1. Maturation of mDC1 cells is associated with induction of CD83 expression and with improved capacity to stimulate mixed lymphocyte responses (MLR) (see, e.g., Zhou and Tedder (1996) Proc Natl. Acad. Sci. USA 93:2588). To study the signal requirements for mDC2 to mature into CD83⁺ cells, we cultured these cells in the presence of anti-CD40 mAbs, LPS plus IFN-γ, or anti-CD40 mAbs, followed by LPS plus IFN-γ. A representative experiment is shown in FIG. 4. A shown in this figure, MDC1 (A) and mDC2 (B) were generated as described above and cultured for a total of seven days. No additional stimuli were added to the control cultures, indicated as (−). LPS (1 ng/ml) plus IFN-γ (10 ng/ml), indicated as (LPS+IFN-γ) in the figure, was added to parallel cultures on day 6 and the cells were harvested on day 7. Another set of the cells was activated with anti-CD40 mAbs (10 μg/ml) on day 5, and the cells were again harvested on day 7, indicated as (αCD40). Alternatively, the cells were activated with anti-CD40 mAbs on day 5, and LPS plus IFN-γ was added on day 6 for an additional 24 hours, indicated as (αCD40/LPS+IFN-γ). The harvested cells were washed and labeled with anti-CDla-FITC and anti-CD83-PE as indicated in FIG. 4. The cells were analyzed by FACScalibur flow cytometer and CellQuest software. Similar data were obtained in five other independent experiments.

When mDC2 cells were cultured in the presence of anti-CD40 mAbs (i.e., pretreated with anti-CD40 mAbs) for 24 hours prior to the addition of LPS and IFN-γ, the majority of the mDC2 differentiated into CD83⁺ cells. Importantly, mDC2 remained CD1a⁻ even upon maturation to CD83⁺ cells (FIG. 4).

Further phenotypic analysis of DC cultured in the presence of LPS plus IFN-γ after pretreatment with anti-CD40 mabs also indicated that mDC1 and mDC2 expressed comparable levels of CD40, CD80, CD86 and MHC class II, while they were CD14⁻ (data not shown), as was also demonstrated for mDC1 and mDC2 cultured in the absence of anti-CD40 mAbs, LPS, and IFN-γ (FIG. 2). In contrast to mDC1, mDC2 did not mature into CD83⁺ DC in the presence of LPS plus IFN-γ (FIG. 4), demonstrating that the signaling requirements for maturation differ between these two DC population subsets. The finding that mDC2 can be matured into CD83⁺ cells, but that the signal requirements of mDC2 for maturation differ from those of mDC1, further indicates that the mDC2 cells of the present invention are phenotypically and functionally distinct from conventional mDC1 cells.

The cytokine production profiles of mature mDC1 and mDC2 were essentially the same as those of the corresponding CD83⁻ population subsets. Regarding IL-12 production, supernatants of mature mDC1 contained 2897±937 picogram/milliliter (pg/ml) IL-12 (mean±SEM), whereas those of mDC2 derived from the same donors contained 125±93 pg/ml IL-12 (n=10). Specifically, in 8 out of 10 experiments, IL-12 production from mature mDC2 was undetectable in ELISA assays in which EL-12 sensitivity is 5 pg/ml. The average of mature mDC2 IL-12 production of 10 experiments was 125±93 pg/ml EL-12 (n=10). The term “substantially lacks IL-12 production,” “substantially lacking in production of L-12,” “substantially decreased production of IL-12,” or “produces substantially no IL-12” in reference to mature mDC2 IL-12 production refers to a substantial decrease or substantial lack in mature mDC2 IL-12 production relative to the mature mDC1 IL-12 production, and typically refers to a mature mDC2 IL-12 production ranging from at least about 50% to about 100% times less, at least about 60% to about 100% times less, at least about 70% to about 100% times less, at least about 80% to about 100% times less, at least about 90% to about 100% times less, at least about 95% to about 100% times less, at least about 97% to about 100% times less, or at least about 99% to about 100% times less, than mature mDC1 IL-12 production.

Regarding IL-10, IL-10 production was undetectable in cultures of mature MDC1 (using the ELISA assays in which IL-10 sensitivity is 5 μg/ml), whereas 215±23 pg/ml (mean±SEM) of IL-10 was produced in the supernatants of CD83⁺ mDC2 (n=4). The term “substantially increased IL-10 production,” “substantially increase in production of IL-10,” “substantially increased production of IL-10,” or “substantially enhanced production of IL-10” in reference to mature mDC2 IL-10 production refers to a substantial increase or substantial enhancement in mature mDC2 IL-10 production relative to the mature mDC1 IL-10 production, and typically refers to a mature mDC2 IL-10 production ranging from at least about 60% to about 100% times greater, at least about 70% to about 100% times greater, at least about 80% to about 100% times greater, at least about 90% to about 100% times greater, at least about 95% to about 100% times greater, at least about 96% to about 100% times greater, at least about 97% to about 99% times greater, or at least about 97% to about 98% times greater, than mature mDC1 IL-10 production.

No significant difference in the levels of IL-6 (n=5) and IL-8 (n=7) in these supernatants was observed (data not shown). Thus, the cytokine production profiles of mDC1 and mDC2 remain stable upon maturation.

### Example 5

### MDC2 Act as Potent Antigen-presenting Cells

Because CD1a may play a role in presentation of antigens at least to CD1restricted T cells (Sieling et al. (1999) J. Immunol. 162:1852), and because the altered cytokine production profile was expected to influence the effector function of the DC, we studied the efficacy of the two DC subsets to induce allogeneic mixed lymphocyte reaction (MLR). The ability of mDC2 to induce an allogeneic MLR was compared to that of mDC1. T cells were purified from peripheral blood mononuclear cells by negatively depleting CD19-, CD 14-, CD16, and CD56-expressing cells using magnetic beads using methods described above and well-known in the art.

MLR was performed using irradiated DC and allogeneic T cells, purified as described above and in Example 1. DC were irradiated (1000 rad) and cultured with allogeneic T cells (1×10⁵ cells/well) in 96-well U-bottom microtiter plates (Costar) at ratios ranging between 1:10 and 1:1250. 1 microCurie (μCi/well) of 3H-thymidine (Amersham, Piscataway, N.J.) was added for the last 16 hours of the cultures, and the cells were harvested onto filter paper using a cell harvester (Tomtec, Hamden, Conn.). ³H-thymidine incorporation was measured using a scintillation counter (MicroBeta, Wallac, Finland) according to procedures well-established in the art.

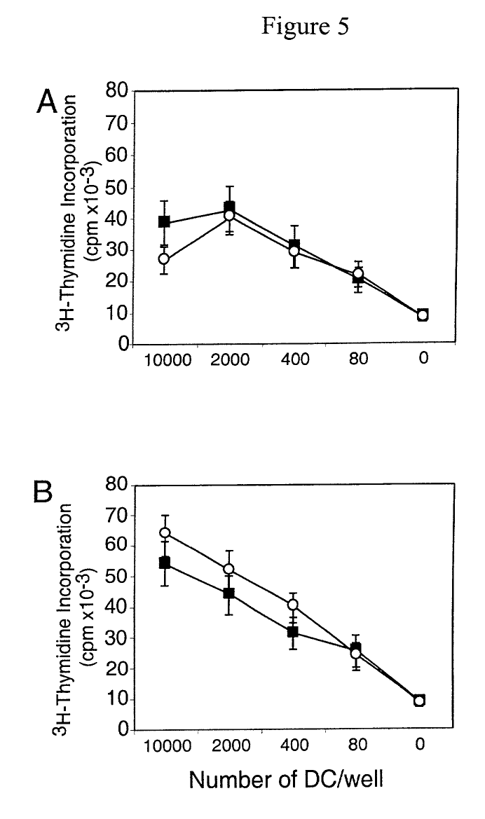

FIG. 5 illustrates the results of the mixed lymphocyte reaction (MLR) induced by immature (panel A) and mature (panel B) mDC1 and mDC2. mDC1 (▪) (closed squares) and mDC2 (∘) (open circles) were generated by culturing peripheral blood monocytes in the presence of IL-4 (400 U/ml) and GM-CSF (800 U/ml) in either RPMI (mDC1) or Yssel's medium (mDC2) for a total of seven days. To generate immature DC (A), no additional stimuli were added, whereas anti-CD40 mAbs (10 μg/ml) were added on day 5, and LPS (1 ng/ml) plus IFN-γ (10 ng/ml) were added on day 6 to generate mature DC (B). DC were irradiated (1000 rad) and cultured with allogeneic purified T cells (1×10⁵ cells/well) at ratios ranging between 1:10 and 1:1250 (DC : T cells) for four days. 1 μCi/well of ³H-thymidine was added for the last 16 hours of the cultures, the cells were harvested, and the ³H-thymidine incorporation was measured by a scintillation counter. The data represent mean±SEM of four separate experiments, each performed in triplicate. As shown in FIG. 5, both mDC1 and mDC2 cells induced potent proliferation of allogeneic T cells. When mature CD83⁺ DC were used as stimulator cells, the responses induced by mDC2 cells generally exceeded those induced by mDC1 cells, especially at high dilution (FIG. 5B), although the differences were not statistically significant. This is consistent with previous studies indicating that the APC function of DC is 4 up-regulated upon maturation (Zhou et al. (1996) J. Immunol. 162:1852). No significant difference in the capacity of mDC1 and mDC2 to induce MLR was observed, irrespective whether the cells expressed CD83 (FIG. 5), indicating that both mDC1 and mDC2 can act as potent APCs.

### Example 6

### Induction of TH0/TH2 Differentiation by MDC2

Exposure to cytokines is known to be a critical influence in the differentiation of T helper cells into Th1 and Th2 subsets. For example, exposure to antigen in the presence of IL12 and IFN-γ leads to the production of Th1 cells, whereas differentiation in the presence of IL-4 results in Th2 cells.

Because of the different cytokine production profiles by mDC1 and mDC2, we speculated that the two subsets would also differ in their capacity to support Th cell differentiation.

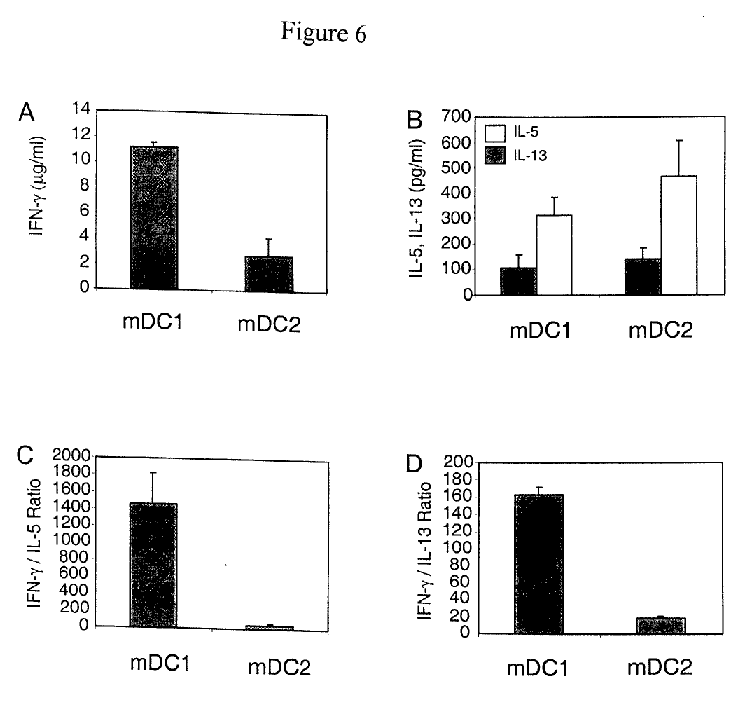

mDC1 and mDC2 were prepared as described above and harvested on day 7, washed, and co-cultured (1×10⁵ cells/well) with purified autologous T cells (1×10⁶ cells/well) in 24-well plates in Yssel's medium. After 5 days of additional culture, T cells were harvested and subsequently stimulated with 1 μg/ml of anti-CD3 mAbs and 10 μg/ml of anti-CD28 mAbs for 24 hours to analyze the cytokine production profiles. The supernatants were then harvested and the concentrations of cytokines were measured by cytokine-specific ELISAs, as described above, in three (IL-5) or four (IFN-γ and IL-13) independent experiments. The results are expressed as mean±SEM. See FIG. 6.

As shown in FIG. 6, conventional DC, i.e., mDC1, skewed Th cell differentiation of Th cells toward Th1 cells producing high levels of IFN-γ, which is consistent with previous studies (see O'Garra (1998) Immunity 27:515). In contrast, T cells cultured in the presence of mDC2 produced significantly less IFN-γ, and the ratio of IFN-γ/IL-5 and IFN-γ/IL13 was consistently higher in cultures activated with conventional mDC1 cells.

IL-4 production was consistently undetectable in supernatants recovered from mDC1/T cell cultures, and the levels were generally low also in cultures of mDC2. However, up to approximately 110 or 111 pg/ml was detected in cultures of mDC2/T cells. Thus, while conventional mDC1 induce differentiation along the Th1 pathway, the mDC2 cells of the present invention are capable of inducing and favor Th0/Th2 differentiation. These data indicate that mDC1 and mDC2 direct the differentiation of Th subsets (or subtypes) with different cytokine production profiles. Because the balance of Th1/Th2 cells is a critical factor in autoimmune disease and in the immune response against pathogens (e.g., Listeria), modulation of the Th1/Th2 balance by the methods of the present invention will be of significant utility in the development of methods for the regulation and therapy of numerous disease states.

### Example 7

### Transfection Efficiencies of MDC2 and MCD1

Because ex vivo transfection of DC followed by in vivo transfer of these cells is an attractive approach in several pharmaceutical applications and immunization protocols (see, e.g., Liu et al. (1998) Nat. Biotechnol. 16:335; Timmerman and Levy (1999) Annu. Rev. Immunol. 50:507), we addressed the question of whether mDC2 can support transgene expression following transfection with conventional expression vectors.

1. Methods for Transfecting DC

The mDC1 and mDC2 cells were transfected after 7 days of culture by electroporation (Gene Pulser, BioRad, Hercules, Calif.). Cells were harvested, washed once, and resuspended in serum-free, antibiotic-free medium (RPMI 1640, Gibco BRL Life Technologies, Rockville, Md.) at a final concentration of 10×10⁶ cells/ml. A total 5×10⁶ DC was mixed with 20 μg of plasmid DNA-encoding green fluorescent protein (GFP) driven by the cytomegalovirus (CMV) immediate-early gene promoter/enhancer (pEGFP-Cl, Clontech, Palo Alto, Calif.) in a 0.4-cm electroporation cuvette. A promoterless vector pEGFP-1 was used as negative control vector (Clontech). Alternatively, the cells were transfected with a vector encoding luciferase (pGL3Control, Promega, Madison, Wis.) or with a promoterless pGL3-Basic (Promega) as a negative control. The cells were subsequently incubated at room temperature (RT) for 1 minute and then subjected to an electric shock of 250 volts (V) and 1050 microFarad (PY) capacitance. The transfected cells were immediately transferred into 3 ml of complete DC culture medium and incubated in 6-well culture plates (Costar) for 24 hours. Alternatively, the cells were transfected using cationic liposomes Lipofectin (Life Technologies; GibcoBRL), Superfect (Qiagen, Valencia, Calif.), DOTAP (N-[1-(2,3-dioleoyloxy)propyl]-N,N,N-trimethylammonium methylsulfate (Boehringer Mannheim) and DOSPER (1,3-di-oleoyloxy-2-(6-carboxyspermyl)propyl-amid (Boehringer Mannheim, Mannheim, Germany) using protocols described previously by Alijagic et al. (1995) “Dendritic cells generated from peripheral blood transfected with human tyrosinase induce specific T cell activation,” Eur. J. Immunol. 25:3100; Manickan et al. (1997) “Enhancement of immune response to naked DNA vaccine by immunization with transfected dendritic cells,” J. Leukoc. Biol. 61:125; and Kronenwett et al. (1998) “Oligodeoxyribonucleotide uptake in primary human hematopoietic cells is enhanced by cationic lipids and depends on the hematopoietic cell subset,” Blood 91:852. The transfection efficiency was evaluated by analyzing GFP expression using a FACScalibur flow cytometer (Becton Dickinson) and Cell Quest software.

2. Results

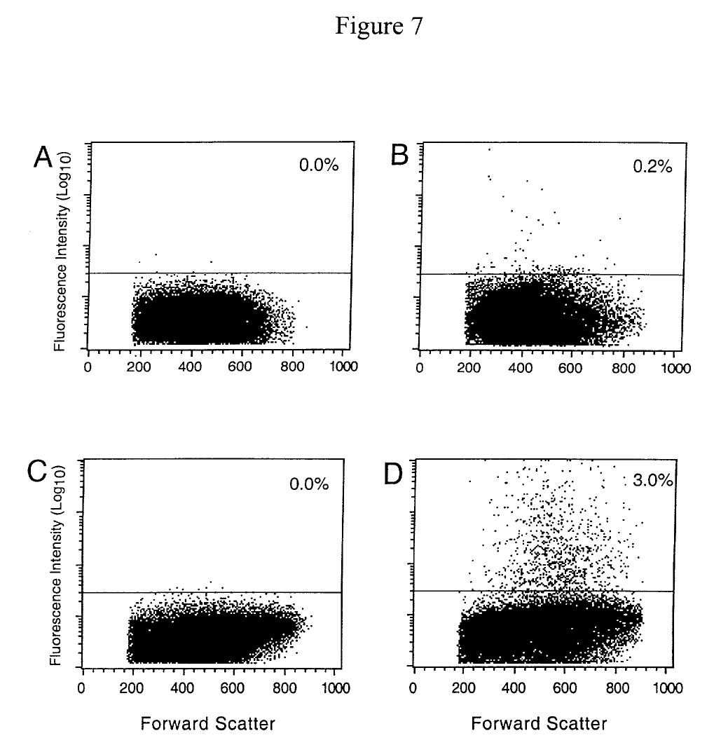

The results of four representative experiments are shown in FIG. 7. In these experiments, susceptibility of mDC1 and mDC2 to transfection by naked DNA vectors (i.e., DNA without transfection-facilitating agents) was examined. A vector-encoding GFP driven by the CMV promoter was transfected into mDC1 and mDC2 cells after 7 days by electroporation, and the level of GFP expression was studied by flow cytometry as described above (see, e.g., Example 1). Further, a total 5×10⁶ DC was mixed with 20 μg of plasmid DNA-encoding GFP driven by the CMV immediate-early promoter/enhancer, or a control vector with no promoter. The cells were subjected to an electric shock of 250 V and 1050 μF capacitance, and incubated in 6-well culture plates for 24 hours. GFP expression was analyzed using a FACScalibur flow cytometer and Cell Quest software.

The transfection efficiency of mDC1 was minimal or absent, ranging between 0.2% and 0.5% in the four separate experiments (mean±SD:0.31±0.17%). However, transfection of mDC2 with the same expression vector under the comparable conditions in parallel experiments resulted in significantly higher frequencies of transfected cells, ranging between 1.3% and 6.9% (mean±SD: 3.5±2.4%) (FIG. 7). The difference in the transfection efficiency between mDC1 and mDC2 is statistically significant (p<0.05, Student's T-test).

Similar results were obtained following transfection with a luciferase-encoding vector. Luciferase expression could not be detected in mDC1 after transfection of a vector encoding the luciferase gene, whereas measurable activity was detected after transfection of the same vector into mDC2 (data not shown). Other transfection methods, such as Lipofectin, Superfect, DOTAP, or DOSPER, did not improve the transfection efficiency of either mDC1 or mDC2 (data not shown). These data indicate that mDC2 are more responsive to transfection than mDC1.

Because conventional dendritic cells (mDC1) are refractory to transfection, their utility in many of in vitro, ex vivo, and in vivo therapeutic and/or prophylactic applications and immunization practices described herein, as well as numerous experimental and pharmaceutical applications that involve, for example, presentation of an uncharacterized antigen. In contrast, given the improved transfection efficiencies of the dendritic cells of the present invention (mDC2), as shown herein, such mDC2 are more useful in applications involving in vitro, ex vivo, or in vivo transfections of dendritic cells.

While the foregoing invention has been described in some detail for purposes of clarity and understanding, it will be clear to one skilled in the art from a reading of this disclosure that various changes in form and detail can be made without departing from the true scope of the invention. For example, all the techniques, methods, compositions, apparatus and systems described above may be used in various combinations. All publications, patents, patent applications, or other documents cited in this application are incorporated by reference in their entirety for all purposes to the same extent as if each individual publication, patent, patent application, or other document were individually indicated to be incorporated by reference in its entirety for all purposes.

What is claimed is:

1. A method of producing a differentiated antigen presenting cell (APC), the method comprising: culturing a population of peripheral blood or bone marrow mononuclear cells in interleukin-4 (IL-4), granulocyte macrophage colony stimulating factor (GM-CSF), and a culture medium comprising insulin, transferrin, linoleic acid, oleic acid, palmitic acid for a sufficient time to produce the differentiated antigen presenting cell.

2. The method of claim 1, wherein the differentiated APC is a dendritic cell.

3. The method of claim 2, wherein the dendritic cell produces substantially no IL-12.

4. The method of claim 3, wherein the dendritic cell produces IL-10.

5. The method of claim 4, wherein the dendritic cell is an mDC2.

6. The method of claim 2, wherein the dendritic cell is a CD1a⁻ dendritic cell.

7. The method of claim 6, wherein the CD1a⁻ dendritic cell is capable of presenting an antigen to a T cell.

8. The method of claim 1, wherein the population of mononuclear cells is derived from a human or a non-human animal.

9. The method of claim 1, further comprising depleting the population of mononuclear cells of T, B and NK cells.

10. The method of claim 9, comprising depleting the population of mononuclear cells with immunomagnetic beads.

11. The method of claim 1, comprising deriving the population of mononuclear cells by density gradient separation of standard buffy coat preparations of peripheral blood.

12. The method of claim 11, further comprising depleting the population of mononuclear cells of T, B and NK cells.

13. The method of claim 12, comprising depleting the population of mononuclear cells with immunomagnetic beads.

14. The method of claim 1, wherein the population of peripheral blood or bone marrow mononuclear cells comprises monocytes.

15. The method of claim 1, wherein the culture medium comprises Iscove's Modified Dulbecco's Medium (IMDM) supplemented with insulin, transferrin, linoleic acid, oleic acid and palmitic acid.

16. The method of claim 15, wherein the culture medium further comprises approximately 0.25% (w/v) bovine serum albumin and between about 1.5 and 2 mg/L 2-amino ethanol.

17. The method of claim 15, wherein the dendritic cell is a CD1a⁻ dendritic cell.

18. The method of claim 17, wherein the dendritic cell substantially lacks L-12 production.

19. The method of claim 18, wherein the dendritic cell has substantially increased IL-10 production as compared to a dendritic cell produced by culturing a population of peripheral blood or bone marrow mononuclear cells in IL-4, GM-CSF, and a culture medium comprising RPMI.

20. The method of claim 6 or 17, wherein the CD1a⁻ dendritic cell induces or promotes Th0/Th2 differentiation of T cells.

21. The method of claim 1, wherein the culture medium comprises Yssel's medium.

22. The method of claim 21, wherein the Yssel's medium further comprises about 10% fetal bovine serum, about 2 milliMolar (mM) glutamine, about 50 Units/milliliter (U/ml) penicillin and about 100 micrograms/milliliter (jig/ml) streptomycin.

23. The method of claim 21, wherein the differentiated APC is a dendritic cell.

24. The method of claim 23, wherein the dendritic cell is a CD1a⁻ dendritic cell.

25. The method of claim 23, wherein the dendritic cell substantially lacks IL-12 production or induces or promote differentiation of T cells to Th0/Th2.

26. The method of claim 23, wherein the dendritic cell has substantially increased IL-10 production as compared to a dendritic cell produced by culturing a population of peripheral blood or bone marrow mononuclear cells in IL-4, GM-CSF, and a culture medium comprising RPMI.

27. The method of claim 23, wherein the CD1a⁻ dendritic cell induces or promotes Th0/Th2 differentiation of T cells.

28. The method of claim 21, further comprising culturing the APC in the presence of an anti-CD40 monoclonal antibody for a period of approximately 24 hours, thereby providing an activated APC; and culturing the activated APC in the presence of lipopolysaccharide (LPS) and interferon-gamma (IFN-γ) for a period of approximately 48 hours, thereby producing a mature CD83⁺, CD1a⁻ dendritic cell.

29. The method of claim 28, wherein the CD83⁺, CD1a⁻ dendritic cell substantially lacks production of IL-12.

30. The method of claim 6 or 24, further comprising introducing to at least one CDla dendritic cell at least one exogenous DNA sequence operably linked to a promoter that is capable of controlling expression of said DNA sequence, which at least one exogenous DNA sequence encodes at least one antigen, in an amount sufficient that expression and presentation of the at least one antigen results, thereby producing an antigen presenting CD1a⁻ dendritic cell.

31. The method of claim 30, further comprising introducing said at least one exogenous DNA sequence to at least one CD1a⁻ dendritic cell by a method selected from electroporation, injection, microinjection, gene gun delivery, lipofection, DOTAP supplemented lipofection, DOSPER supplemented lipofection, or Superfection.

32. The method of claim 6 or 24, further comprising introducing a sufficient amount of at least one antigen or fragment thereof to at least one CD1a⁻ dendritic cell, such that presentation of the at least one antigen on least one CD1a⁻0 dendritic cell occurs, thereby producing an antigen presenting CD1a⁻ dendritic cell.

33. A differentiated antigen presenting cell (APC), which differentiated APC does not express CDla cell surface marker.

34. The differentiated APC of claim 33, wherein said differentiated APC comprises a monocyte-derived CD1a⁻ dendritic cell.

35. The differentiated APC of claim 34, wherein monocyte-derived CD1a⁻ dendritic cell substantially lacks IL-12 production.

36. The differentiated APC of claim 34, wherein the monocyte-derived CD1a⁻ dendritic cell induces or promotes differentiation of T cells to Th0/Th2 subtypes.

37. The differentiated APC of claim 34, wherein the monocyte-derived CD1a⁻ dendritic cell is produced by culturing a population of monocytes in interleukin-4 (IL-4), granulocyte macrophage colony stimulating factor (GM-CSF), and a culture medium comprising Iscove's Modified Dulbecco's Medium (IMDM) supplemented with insulin, transferrin, linoleic acid, oleic acid and palmitic acid.

38. The differentiated APC of claim 37, wherein the culture medium comprises Yssel's medium.

39. The differentiated APC of claim 37, wherein the monocyte-derived CD1a⁻ dendritic cell has substantially increased IL-10 production as compared to a dendritic cell produced by culturing a population of peripheral blood or bone marrow mononuclear cells in IL4, GM-CSF, and a culture medium comprising RPMI.

40. The differentiated APC of claim 34, wherein the monocyte-derived CD1a⁻ dendritic cell comprises an mDC2.

41. The differentiated APC of claim 34, wherein the monocyte-derived CD1a⁻ dendritic cell has a transfection efficiency greater than that of a dendritic cell produced by culturing a population of monocytes in IL-4, GM-CSF, and a culture medium comprising RPMI.

42. A method of inducing in a subject an immune response to at least one antigen, said method comprising administering to the subject a population of CD1a⁻ dendritic cells, said CD1a⁻ dendritic cells presenting at least one of said at least one antigen, in an amount sufficient to induce the immune response to said at least one antigen.

43. The method of claim 42, wherein said CD1a⁻ dendritic cell substantially lacks EL-12 production.

44. The method of claim 42, wherein said CD1a⁻ dendritic cell is produced by culturing a population of peripheral blood or bone marrow mononuclear cells in interleukin-4 (IL-4), granulocyte macrophage colony stimulating factor (GM-CSF), and a culture medium comprising insulin, transferrin, linoleic acid, oleic acid, palmitic acid for a sufficient time to produce the differentiated antigen presenting cell.

45. The method of claim 42, wherein the subject is a human or a non-human animal.

46. A method of inducing differentiation of T cells, the method comprising: co-culturing a population of T cells with population of CD1a⁻ antigen presenting cells (APC), thereby inducing or promoting differentiation of said T cells.

47. The method of claim 46, wherein the T cells comprise naive T cells.

48. The method of claim 46, wherein the antigen presenting cell is a CD1a⁻ dendritic cell.

49. The method of claim 48, wherein the CD1a⁻ dendritic cell produces substantially no IL-12.

50. The method of claim 3 or 48, wherein the dendritic cell produces substantially no IL-12 compared to a dendritic cell produced by culturing a population of peripheral blood or bone marrow mononuclear cells in IL-4, GM-CSF, and a culture medium comprising RPMI.

51. A differentiated T cell produced by the method of claim 46.

52. A composition comprising CD1a⁻ dendritic cells.

53. The composition of claim 52, wherein said CDla dendritic cells are capable of presenting an antigen to a T cell.

54. The composition of claim 52, wherein said CDla dendritic cells produce substantially no IL-12.

55. The composition of claim 52, wherein said CD1a⁻ dendritic cells promote differentiation of T cells to a Th0/Th2 subtype.

56. The composition of claim 52, wherein said CD1a⁻ dendritic cells display or present at least one antigen or antigenic fragment thereof.

57. The composition of claim 56, wherein the at least one antigen or antigenic fragment comprises a protein or peptide differentially expressed on a cell selected from the group consisting of a tumor cell, a bacterially-infected cell, a parasitically-infected cell, and a virally-infected cell, a target cell of an autoimmune response.

58. The composition of claim 52, wherein the composition comprises a vaccine.

59. The composition of claim 52, further comprising a pharmaceutically acceptable carrier.

60. A method of inducing or modulating an immune response in an immunocompromised subject, said method comprising administering to the subject a population of CD1a⁻ dendritic cells in an amount sufficient to induce or modulate an immune response in the subject.

61. An ex vivo method of inducing in a subject a therapeutic or prophylactic immune response against at least one antigen, the method comprising:
a) culturing a population of monocytes obtained from the subject with IL-4, GM-CSF, and a culture medium comprising Iscove's Modified Dulbecco's Medium (IMDM) supplemented with insulin, transferrin, linoleic acid, oleic acid and palmitic acid for a sufficient time to produce a population of dendritic cells comprising CD1a⁻ dendritic cells;
b) introducing to the population of CD1a⁻ dendritic cells a sufficient amount of at least one antigen, or a sufficient amount of an exogenous DNA sequence operably linked to a promoter that controls expression of said DNA sequence, said DNA sequence encoding at least one or said at least one antigen, such that the presentation of the antigen on the CD1a⁻ dendritic cells results; and
c) administering the antigen-presenting CD1a⁻ dendritic cells to the subject in an amount sufficient to induce a therapeutic or prophylactic immune response against said at least one antigen.

62. The method of claim 61, wherein the culture medium comprises Yssel's medium.

63. A method for therapeutically or prophylactically treating a disease in a subject suffering from said disease, the method comprising:
a) culturing a population of monocytes obtained from the subject with IL-4, GM-CSF, and a culture medium comprising Iscove's Modified Dulbecco's Medium (IMDM) supplemented with insulin, transferrin, linoleic acid, oleic acid and palmitic acid for a sufficient time to produce a population of CD1a⁻ dendritic cells;
b) introducing to the population of CD1a⁻ dendritic cells a sufficient amount of at least one disease-associated antigen, or a sufficient amount of an exogenous DNA sequence operably linked to a promoter that controls expression of said DNA sequence, said DNA sequence encoding at least one of said at least one disease-associated antigen, such that presentation of the disease-associated antigen on the CD1a⁻ dendritic cells results; and
c) administering a therapeutic or prophylactic amount of the CD1a⁻ dendritic cells presenting the disease-associated antigen to the subject to treat said disease.

64. The method of claim 63, wherein the culture medium comprises Yssel's medium.

65. A method for therapeutically or prophylactically treating a disease in a subject suffering from the disease, the method comprising:
a) culturing a population of monocytes obtained from the subject with IL-4, GM-CSF, and a culture medium comprising Iscove's Modified Dulbecco's Medium (IMDM) supplemented with insulin, transferrin, linoleic acid, oleic acid and palmitic acid for a sufficient time to produce a population of CD1a⁻ dendritic cells;
b) contacting the population of CD1a⁻ dendritic cells with a population of diseased cells from a tissue or organ of the subject, thereby inducing presentation of a disease-associated antigen on the CD1a⁻ dendritic cells; and
c) administering a therapeutic or prophylactic amount of CD1a⁻ dendritic cells presenting the disease-associated antigen to the subject to treat the disease.

66. The method of claim 63, wherein the culture medium comprises Yssel's medium.

67. The method of claim 63, wherein the disease is a cancer.

68. A monocyte-derived dendritic cell, wherein the dendritic cell does not express a CD1a cell marker, substantially lacks EL-12 production, produced IL-1, and promotes Th0/Th2 lineage differentiation of T cells.

69. A monocyte-derived dendritic cell produced by culturing a population of monocyte cells in interleukin-4 (IL-4), granulocyte macrophage coloby stimulating factor (GM-CSF), and a culture medium comprising insulin, transferrin, linoleic acid, oleic acid, and palmitic acid, wherein the monocyte-derived dendritic cell has an altered cytokine profile compared to a dendritic cell produced by culturing a population of monocyte cells in IL-4, GM-CSF, and a culture medium comprising RPMI.
